SEQUENCE LISTING (37 sequences)

SEQ ID NO: 1 - DNA, 354 bp, Homo sapiens
  source 1..354: mol_type = unassigned DNA
gaggtgcagctgttggagtctgggggaggcgtggtccagcctgggaggtccctgagactctcctgtatagcctctggattcaccttcagtagctatcctatgacctgggtccgccaggctccaggcaaggggctggagtgggtggcaagtatatcatatgacggaagttataaatataaggtagactccatgaagggccgactcaccatctccagagacaattccaagaacacgctgtatttggaaatgaacagcctgacagctgaggacacggctgtgtattactgtgcgaggacagctttctttaacgcctatgacttctggggccagggaaccctggtcaccgtctcctca

SEQ ID NO: 2 - protein, 118 aa, Homo sapiens
EVQLLESGGGVVQPGRSLRLSCIASGFTFSSYPMTWVRQAPGKGLEWVASISYDGSYKYKVDSMKGRLTISRDNSKNTLYLEMNSLTAEDTAVYYCARTAFFNAYDFWGQGTLVTVSS

SEQ ID NO: 3 - DNA, 324 bp, Homo sapiens
  source 1..324: mol_type = unassigned DNA
gaaattgtgatgacgcagtctccagccaccctgtctgtgtctccaggggaaagagccaccctctcctgcagggccagtcagagtgttagaagcaacttagcctggtaccagcagaaacctggccaggctcccaggctcctcatctatgctgcatccaccagggccactggtatcccagccaggttcagtggcagtgggtctgggacagagttcactctcaccatcagcagcctgcagtctgaagatttcgcagtttattactgtcagcagtataatgaatggtttcggacgttcggccaagggaccaaggtggaaatcaaacga

SEQ ID NO: 4 - protein, 108 aa, Homo sapiens
EIVMTQSPATLSVSPGERATLSCRASQSVRSNLAWYQQKPGQAPRLLIYAASTRATGIPARFSGSGSGTEFTLTISSLQSEDFAVYYCQQYNEWFRTFGQGTKVEIKR

SEQ ID NO: 5 - DNA, 345 bp, Homo sapiens
  source 1..345: mol_type = unassigned DNA
caggtgcagctgcaggagtcgggcccaggactggtgaaggctgcggagaccctgtccctcacctgcactgtctcccgtggctccttcagtgattacttctggaattggttccggcagcccgccgggaagcgcctggagtggcttgggcgtgtctataccagtggaagtgtcgactacaacccctccctcaagagtcgagtcaccgtgtcagtggacacgtccaagaagcagttctccctgaggctgagctctgtgaccgtcgcggacacggccgtgtattattgtgcgagaggactgtccggttttgactactggggccagggagccctggtcaccgtctcccca

SEQ ID NO: 6 - protein, 115 aa, Homo sapiens
QVQLQESGPGLVKAAETLSLTCTVSRGSFSDYFWNWFRQPAGKRLEWLGRVYTSGSVDYNPSLKSRVTVSVDTSKKQFSLRLSSVTVADTAVYYCARGLSGFDYWGQGALVTVSP

SEQ ID NO: 7 - DNA, 324 bp, Homo sapiens; identical to SEQ ID NO: 3
  source 1..324: mol_type = unassigned DNA

SEQ ID NO: 8 - protein, 108 aa, Homo sapiens; identical to SEQ ID NO: 4

SEQ ID NO: 9 - DNA, 345 bp, Homo sapiens; identical to SEQ ID NO: 5
  source 1..345: mol_type = unassigned DNA

SEQ ID NO: 10 - protein, 115 aa, Homo sapiens; identical to SEQ ID NO: 6

SEQ ID NO: 11 - DNA, 324 bp, Homo sapiens
  source 1..324: mol_type = unassigned DNA
gacatccagatgacccagtctccatcttccgtgtctgcatctgtgggagacagagtcaccgtcacttgtcgggcgagtcagggtattagcagttggttagcctggtatcagcagaaaccagggaaagcccctaaactcctgatccatgctgcatccagtttgcaaagtggggtcccatcaaggttcatcggcagtggatctgggacagatttcactctcaccatcaccagcctgcaggctgaagattttgcaacctactattgtcaacaggctgacagtctcccttttactttcggcggagggaccaaggtggacttcaaacga

SEQ ID NO: 12 - protein, 108 aa, Homo sapiens
DIQMTQSPSSVSASVGDRVTVTCRASQGISSWLAWYQQKPGKAPKLLIHAASSLQSGVPSRFIGSGSGTDFTLTITSLQAEDFATYYCQQADSLPFTFGGGTKVDFKR

SEQ ID NO: 13 - DNA, 363 bp, Homo sapiens
  source 1..363: mol_type = unassigned DNA
gaggtgcagctggtggagtcggggggaggcttggtacagcctggggggtccctgagactctcctgtgcagcctctggattcacctttagcagctatgccatgagctgggtccgccaggctccagggaaggggctggagtgggtctcaggtataagtgctcgtggtggtagcacatactacgcagactccgtgaagggccggttcaccatctccagagacaattccaagaacacgctgtatctccaaatgaacagcttgagagccgaggacacggccgtatattactgtgcgaaagataggggcaggatagcagcagctcactttgactactggggccagggaaccctggtcaccgtctcctca

SEQ ID NO: 14 - protein, 121 aa, Homo sapiens
EVQLVESGGGLVQPGGSLRLSCAASGFTFSSYAMSWVRQAPGKGLEWVSGISARGGSTYYADSVKGRFTISRDNSKNTLYLQMNSLRAEDTAVYYCAKDRGRIAAAHFDYWGQGTLVTVSS

SEQ ID NO: 15 - DNA, 324 bp, Homo sapiens; identical to SEQ ID NO: 11
  source 1..324: mol_type = unassigned DNA

SEQ ID NO: 16 - protein, 108 aa, Homo sapiens; identical to SEQ ID NO: 12

SEQ ID NO: 17 - DNA, 348 bp, Homo sapiens
  source 1..348: mol_type = unassigned DNA
gaggtgcagctggtggagtcaggcccaggactggtgaagccttcggagaccctgtccctcacctgcactgtctcttatgactccatcaataattactactggagctggatccggcagcccgccgggaagggactggagtggattgggcgtatctatcccggtgggagcaccaagtacaacccctccctcaagagtcgagtcaccatgtcagtagacacgtccaagaaccagttctccctgaagctgagttctgtgaccgccgcggacacggccgtgtattactgtgcgagagggggtttcggggagtttgactactggggccagggaaccctggtcaccgtctcctca

SEQ ID NO: 18 - protein, 116 aa, Homo sapiens
EVQLVESGPGLVKPSETLSLTCTVSYDSINNYYWSWIRQPAGKGLEWIGRIYPGGSTKYNPSLKSRVTMSVDTSKNQFSLKLSSVTAADTAVYYCARGGFGEFDYWGQGTLVTVSS

SEQ ID NO: 19 - DNA, 324 bp, Homo sapiens; identical to SEQ ID NO: 11
  source 1..324: mol_type = unassigned DNA

SEQ ID NO: 20 - protein, 108 aa, Homo sapiens; identical to SEQ ID NO: 12

SEQ ID NO: 21 - DNA, 354 bp, Homo sapiens; identical to SEQ ID NO: 1
  source 1..354: mol_type = unassigned DNA

SEQ ID NO: 22 - protein, 118 aa, Homo sapiens; identical to SEQ ID NO: 2

SEQ ID NO: 23 - DNA, 324 bp, Homo sapiens; identical to SEQ ID NO: 11
  source 1..324: mol_type = unassigned DNA

SEQ ID NO: 24 - protein, 108 aa, Homo sapiens; identical to SEQ ID NO: 12

SEQ ID NO: 25 - DNA, 363 bp, Homo sapiens; identical to SEQ ID NO: 13
  source 1..363: mol_type = unassigned DNA

SEQ ID NO: 26 - protein, 121 aa, Homo sapiens; identical to SEQ ID NO: 14

SEQ ID NO: 27 - DNA, 324 bp, Homo sapiens; identical to SEQ ID NO: 3
  source 1..324: mol_type = unassigned DNA

SEQ ID NO: 28 - protein, 108 aa, Homo sapiens; identical to SEQ ID NO: 4

SEQ ID NO: 29 - DNA, 348 bp, Homo sapiens; identical to SEQ ID NO: 17
  source 1..348: mol_type = unassigned DNA

SEQ ID NO: 30 - protein, 116 aa, Homo sapiens; identical to SEQ ID NO: 18

SEQ ID NO: 31 - DNA, 324 bp, Homo sapiens; identical to SEQ ID NO: 3
  source 1..324: mol_type = unassigned DNA

SEQ ID NO: 32 - protein, 108 aa, Homo sapiens; identical to SEQ ID NO: 4

SEQ ID NO: 33 - DNA, 363 bp, Homo sapiens; identical to SEQ ID NO: 13
  source 1..363: mol_type = unassigned DNA

SEQ ID NO: 34 - protein, 121 aa, Homo sapiens; identical to SEQ ID NO: 14

SEQ ID NO: 35 - DNA, 327 bp, Homo sapiens
  source 1..327: mol_type = unassigned DNA
ggaattgtgttgacccagtctccaggcaccctgtctttgtcttcaggggaaggagccaccctctcctgcagggccagtcagagtgttagcaacggccaattaacctggtaccagcagaagcctggccaggctcccaggctcctcatctatggtgcatccagcagggccactggcatcccagacaggttcagtggcagtgggtctgggacagacttcactctcaccatcagcagactggagcctgaagattttgcagtgtattactgtcaccagtatgatggctccccggagacgttcggccaagggaccaaggtggaaatcaatcga

SEQ ID NO: 36 - protein, 109 aa, Homo sapiens
GIVLTQSPGTLSLSSGEGATLSCRASQSVSNGQLTWYQQKPGQAPRLLIYGASSRATGIPDRFSGSGSGTDFTLTISRLEPEDFAVYYCHQYDGSPETFGQGTKVEINR

SEQ ID NO: 37 - DNA, 3758 bp, Homo sapiens
  source 1..3758: mol_type = unassigned DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctactagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggcggccgccatggcgacgggttcaagaacttccctacttcttgcatttggcctgctttgtttgccgtggttacaggagggctcggcaagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtcgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagtggccgctcccagcgtgttcatcttccctccctctgatgaacagctgaaaagcggaacagccagcgtggtgtgtctgctgaacaacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctaaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2025/0101082 A1
(43) Pub. Date: Mar. 27, 2025

(54) METHODS FOR ELIMINATING HEPATITIS B VIRUS CCCDNA AND RCDNA AND THE HEPATITIS B DRUGS USED IN THE METHODS THEREOF

(71) Applicant: HBVtech, LLC, Frederick, MD (US)

(72) Inventor: Yong-yuan ZHANG, Germantown, MD (US)

(73) Assignee: HBVtech, LLC, Frederick, MD (US)

(21) Appl. No.: 18/721,435

(22) PCT Filed: Dec. 21, 2022
(86) PCT No.: PCT/US2022/081966

§ 371 Date: Jun. 18, 2024

Related U.S. Application Data
(60) Provisional application No. 63/265,816, filed on Dec. 21, 2021.
(60) Provisional application No. 63/382,091, filed on Nov. 2, 2022.

(51) Int. Cl. C07K 16/08; A61K 48/00; A61P 31/20

(52) U.S. Cl. CPC C07K 16/082; A61K 48/0033; A61P 31/20

(57) ABSTRACT

Disclosed herein is a method of treating human chronic hepatitis B virus infection by reducing or eliminating cccDNA in liver cell of an HBV infected patient for 3 months or longer.

### BACKGROUND

Hepatitis B virus (HBV) has chronically infected 302 million people worldwide and nearly one million die of HBV related diseases each year. Current HBV drugs rarely deliver durable efficacy after years of medication, let alone HBV cure.

HBV covalently closed circular DNA (cccDNA) is formed in the nuclei of infected cells upon infection and serves as transcriptional template (Nassal M, Gut 64, 1972-1984 (2015); Tuttleman et al., Cell 47, 451-460 (1986)). The presence of cccDNA is viewed as the root cause for persistent HBV infection. Current HBV cure strategy aims to directly eliminate or permanently silence cccDNA (Alter et al., Hepatology 67, 1127-1131 (2018)). Drugs which can directly eliminate or permanently silence cccDNA remain elusive.

Clinical evidence shows frequent serum viral population turnover, for instance, pre-C, core, pre-S, S, or drug resistant mutants frequently replace wild type (WT) viruses in chronic HBV infection (Brunetto et al., Proc Natl Acad Sci U S A 88, 4186-4190 (1991); Carman et al., Lancet 2, 588-591 (1989).; Lok et al., Proc Natl Acad Sci U S A 91, 4077-4081 (1994).; Okamoto et al., J Virol 68, 8102-8110 (1994).; Zoulim and Locarnini, Proc Natl Acad Sci USA 91, 4077-4081 (1994)), suggesting frequent cccDNA loss and replacement (turnover) in the livers, and further suggesting that: 1. Initial viral population is frequently cleared, and 2. there are new rounds of infection upon clearing the initial viral populations in the infected livers.

An effective method for treating chronic HBV infection is needed. In our previously invention (U.S. Pat. No. 11,136,378B2), it was disclosed that chronic HBV infection can be treated by administering to the HBV infected human patient HBV neutralizing antibodies or HBV therapeutic vectors expressing such HBV neutralizing antibodies at a level that is higher than the level of the serum HBV particles in the HBV infected human patient, such that level of HBV neutralizing antibodies results in a level of undetectable HBV particles in serum or a full HBsAg seroconversion from HBsAg positive to anti-HBs antibody positive. Further research was conducted, and new discoveries are described in this application.

### SUMMARY OF INVENTION

This invention discloses how to effectively cure chronic hepatitis B infection and how to simplify curative treatment and shorten treatment course. Some of the discoveries upon which the present invention is built comprises following elements:
1. In contrast to current consensus in HBV field, clearing HBV infection does not require directly targeting or permanently silencing HBV cccDNA;
2. Further in contrast to the consensus in HBV field, clearing HBV infection does not require specific HBV cellular immunity either;
3. Reducing and eliminating cccDNA from infected livers of HBV infected patients through blocking replenishing HBV rcDNA pool;
4. Blocking rcDNA replenishment through blocking new rounds of infection with sustained high level of anti-HBs antibodies;
5. Providing AAV vectors-based HBV drugs that endogenously express sustained high level of anti-HBs antibodies after injection into muscle cells, which are independent of host's adaptive immunity;
6. Most chronic HBV infected patients can be treated through a single injection of AAV vector-based HBV drugs;
7. A completely sustained anti-HBs antibody seroconversion, that is a change from HBsAg positive and anti-HBs antibody negative (HBsAg+/anti-HBS−) to HBsAg negative and anti-HBs positive (HBsAg−/anti-HBS+), is required for achieving a more effective HBV cure;
8. The most effective way to reduce serum HBsAg to undetectable level (HBsAg seroclearance) is to achieve a complete sustained anti-HBs seroconversion;
9. The current lifetime daily medication-based HBV treatment can be shortened to a finite period when it is added or combined with AAV-anti-HBs vectors that express sustained high level of anti-HBs antibody; and
10. The current therapies mediated or/and spontaneous clearance induced HBsAg seroclearance or HBV functional cure are often reversed, i.e. non-durable. However this can be made durable by adding AAV-anti-HBs vector or giving exogenous anti-HBs antibodies.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 shows the kinetic serum antibody level in malaria group (A, n=5) and anti-HBS group (B, n=15). AAV vectors that express either malaria antibody or anti-HBs antibody, were intramuscularly injected at day 49 post infection (pi) at a dose of 10¹¹ genome copies and kinetic serum antibody levels determined by ELISA, are shown from day 49 to day 183 pi. It's to be noted that two test animal No. 970 and 909 in anti-HBs group were intraperitoneally injected with mouse anti-HBs as control (250 μg per injection triweekly for 9 times). No antibody detected at day 49 and 0.1 μg/ml assigned for plotting curves on log scale.

FIG. 2 shows that viremia and serum HBsAg level were significantly lowered in anti-HBs treated group. FIG. 2A shows the different viremic kinetics between malaria antibody and three anti-HBs groups. Anti-HBS-Total include all 15 mice treated with anti-HBs antibodies. The 15 mice were further divided into two subgroups: Anti-HBS-A group consists of 9 mice and their infection reached the peak before day 183 and anti-HBS-B group comprises 6 animals and their infection was markedly retarded and not yet reached the peak. Viremia level in anti-HBS-B group was lowered by >100-fold over several timepoints compared to malaria group. FIG. 2B shows that the average viremia (black bars) at day 183 was significantly lowered in both anti-HBs-Total and anti-HBs-B groups. Average serum HBsAg level (gray curve) was significantly lowered in all 3 anti-HBs groups compared to malaria group at day 183.

FIG. 3 shows HBV functional cure in animal 970. FIG. 3A shows both viremia and serum HBsAg became undetectable after starting anti-HBs treatment. The lower limit of detection for serum HBV DNA is 100 copies/ml and 1 ng/ml for serum HBsAg and anti-HBS antibody, respectively. Comparable viremia to pre-treatment level of 970 from animal 973 is also plotted as a reference to pre-treatment level of intrahepatic HBV DNA in FIG. 3B. FIG. 3B shows that the average intrahepatic rcDNA levels (copies/cell) from two rounds of 20 liver samplings in 970 were significantly lowered by 500 to 1,800-fold (p=1.33E-9 and 1.29E-9), and average cccDNA level was 100-675-fold lowered (p=5.27E-7 and 4.69E-7) than animal 973, whose rcDNA and cccDNA levels serve as pre-treatment level control. FIG. 3C shows that rcDNA ≤1 copy/cell were detected in 7 of 20 liver samplings of the 1st round and 20 of 20 samplings of the 2^{nd} round (FIG. 3C2). No cccDNA was detected in 3 of 20 samplings of the 1^{st} round and in 7 of 20 samplings of the 2^{nd} round. Very low cccDNA level detected in most cccDNA samplings and no cccDNA amplification detected in any of 40 samplings (FIGS. 3C1 and C2).

FIG. 4 shows the diverse cccDNA levels and cccDNA loss in a fraction of infected cells in HBV infected human livers of chimeric mice. Up to 60-fold difference in cccDNA level among different samplings detected despite comparable rcDNA level among the corresponding liver samplings of 907 liver (FIG. 4A). More than 30-fold difference in cccDNA level was detected among different samplings despite comparable rcDNA level among the corresponding liver samplings of animal 909 liver (FIG. 4B). cccDNA level ≤1 copy/cell was detected in 5 of 20 samplings in animal 907 (FIG. 4A) and in 17 of 20 samplings in animal 909 (FIG. 4B) and was detected in 260 (46%) of total 566 cccDNA samplings (FIG. 4C). The number of liver samplings with average cccDNA level ≤1 copy/cell was significantly higher in anti-HBs group than malaria antibody group (X²=25.2 and p<0.001) (FIG. 4D).

FIG. 5 shows that the average intrahepatic rcDNA level was lowered by 4,000 to 10,000 copies/cell in all 9 liver samples treated with anti-HBs antibodies compared to animal 907 treated with malaria antibody. Average rcDNA level was calculated from 20 samplings of each liver sample. The efficiency to lower rcDNA level is largely viremia level dependent (FIG. 5A). P values ranged between E-9 and E-16 (FIG. 5B).

FIG. 6 illustrates the three components responsible for lowering intrahepatic rcDNA level with anti-HBs therapy. FIG. 6A illustrates the virion secretion that vacates rcDNA from infected cells but existing cccDNA replenishes rcDNA. FIG. 6B illustrates the spontaneous cccDNA loss. A combination of virion secretion with cccDNA loss can clear HBV from infected cells but not durably in absence of anti-HBs. FIG. 6C illustrates the blocking of new rounds of infection with anti-HBs establishes durable HBV clearance.

FIG. 7 shows the major animal experimental procedures, timing, and duration. N is the number of animals at 3 timepoints.

FIG. 8 shows the differences in HBV infection level between animal 907 and 973. Animal 907 was treated with AAV vector expressing malaria antibody while animal 973 was treated with AAV vector expressing anti-HBs antibody. FIG. 8A shows the differences in viremia are >100-fold at late timepoints. FIG. 8B shows the significant differences in rcDNA and cccDNA level detected between two animals.

FIG. 9 shows major experimental procedures and timelines of HBV infected uPA/SCID chimeric mice treated with 9-week of entecavir, subsequently added with AAV-anti-HBS vectors.

FIG. 10 shows that on the adding of AAV-anti-HBs vectors to 9-week ETV treatment prevented HBV relapse and the boosted anti-HBs antibody levelled to HBsAg loss/a complete anti-HBs seroconversion and HBV functional cure. FIG. 10A shows kinetic viremia curves in untreated (blue) and ETV monotherapy (orange) group. FIG. 10B shows kinetic serum HBsAg curves in untreated and ETV only groups. FIG. 10C and FIG. 10D show kinetic viremia and serum HBsAg curves among ETV treated 10 animals with add-on. FIG. 10E shows the number of animals with no HBV relapse and undetectable HBsAg. FIG. 10F shows the number of animals with HBV functional cure.

FIG. 11 shows that HBV functional cure occurred upon or after a complete sustained anti-HBs seroconversion that was achieved with boosting anti-HBs antibody level at different timepoints. FIG. 11A-E shows HBV functional cure markers established upon or after anti-HBs seroconversion. FIG. 11F shows that the serum HBV DNA remained detectable at the end of experiment featured with HBsAg−/HBV DNA+/anti-HBS+ or a pre-cure phase.

FIG. 12 shows that HBV relapse is caused by new rounds of infection. FIG. 12A shows that HBV relapse can't be prevented by anti-HBs treatment if cccDNA persists in infected cells. FIG. 12B. HBV relapse prevented with anti-HBs treatment because there is/are spontaneous cccDNA loss and new rounds of infection after ETV withdrawal. FIG. 12C shows that it takes years/decades to reach a complete anti-HBs seroconversion in most spontaneously cured cases that are mainly driven by reducing HBsAg level. FIG. 12D shows that sufficient anti-HBs antibody therapy can quickly realize a complete anti-HBsAg seroconversion, reducing the duration required for HBV functional cure.

### DETAILED DESCRIPTION

Embodiments identified herein as exemplary are intended to be illustrative and not limiting. Tables 1 and 2 outline some main inventive features of the present invention and they are further elaborated in the description below.

TABLE 1
Some Inventive Features of the Present Invention

| | | Current HBV drugs and |
|---|---|---|
| | Present Invention | treatment |
| How is chronic HBV | By new rounds of infection | By longevity of cccDNA |
| infection maintained | | molecules |
| Treatment targets | Extracellular viral particles | Intracellular replication steps |
| Method for eliminating | Blocking cccDNA | Inhibiting cccDNA synthesis |
| cccDNA and delivering HBV | replenishment through | and/or killing infected cells |
| cure | depleting rcDNA | |
| Method for depleting rcDNA | Blocking rcDNA replenishment | Intracellular inhibition of HBV |
| | | replication |
| Method for blocking rcDNA | Blocking new rounds of | Intracellular inhibition of HBV |
| replenishment | infection | replication |
| Which arm of immunity has | Specific humoral immunity | Specific cellular immunity to |
| to be expanded for HBV cure | to HBsAg | HBV |
| Method for expanding host's | Engineered humoral | Traditional therapeutic |
| immunity | immunology or gene therapy | vaccines depend upon host's |
| | independent of adaptive | adaptive immunity using |
| | immunity using non- | traditional immune cells |
| | immune cells | |
| Method for effectively | A complete and sustained | Inhibiting intracellular HBsAg |
| reducing and clearing serum | anti-HBs seroconversion | synthesis or preventing HBsAg |
| HBsAg for HBV cure | that protects cells cleared of | secretion from infected cells |
| | HBV from reinfection | |
| Method for increasing anti- | Endogenously and may only | Exogenously and repeated |
| HBs level | require a single injection | infusions required |
| Treatment regimens | Only require a definite | Daily medication and infinite |
| | treatment duration and may | treatment duration |
| | only require a single | |
| | injection | |

The present invention further describes the methods to alter HBV infection status through interventionally elevating serum anti-HBs antibody level as outlined in Table 2.

TABLE 2
Elevating serum anti-HBs level alters HBV infection outcomes and serologic markers

| Anti-HBs level | Absent | ≤Serum HBsAg level | >Serum HBsAg level |
|---|---|---|---|
| Serologic markers | HBsAg+/anti-HBs− | HBsAg+/anti-HBs+ | HBsAg−/anti-HBS+ |
| HBV infection status | HBV infection ongoing or HBV | Partially blocking new rounds of | HBsAg loss/anti-HBS |
| | relapse after treatment stopped | infection leading to reducing | seroconversion and |
| | | HBV infection level or no relapse | functional cure |
| | | after treatment stopped | |

In one embodiment, in contrast to the established consensus in HBV field that the exhausted specific cellular immunity is responsible for chronic HBV infection, the present invention emphasizes that a high level of specific humoral immunity to HBsAg is required for curing hepatitis B.

In one embodiment, the present invention distinguishes from current ultimate treatment targets that aim to directly inhibits HBV cccDNA synthesis or kill HBV infected cells through specific HBV cellular immunity for cccDNA elimination. This invention devises a method for reducing and eliminating HBV cccDNA without directly inhibiting cccDNA synthesis or killing infected cells.

In one expanding embodiment, the present invention comprises two principles in formulating cccDNA elimination method: 1. takes advantage of spontaneous HBV clearance including spontaneous cccDNA loss from HBV infected cells; and 2. blocks cccDNA replenishment.

In one embodiment, the present invention emphasizes blocking cccDNA replenishment should be mediated through depleting HBV rcDNA.

In one embodiment, the present invention emphasizes depleting HBV rcDNA includes blocking rcDNA replenishment.

In one embodiment, the present invention emphasizes blocking rcDNA replenishment mainly requires blocking new rounds of infection.

Current HBV treatment methods for HBV drugs which have been approved or under development focus on the inhibition of intracellular HBV replication and do not aim to directly increase HBV neutralizing antibody level, which is disclosed in the present invention.

Some biopharma companies are developing exogenous HBV neutralizing antibodies. However, they attribute the observed efficacy to Fc receptors mediated cellular functions (Lampp, Hepatology 74, 513A (2021); Zhang et al., Gut 65, 658-671 (2016).), not the function of direct neutralization even though regular anti-HBs antibodies without introducing the mutations in the Fc portion of human IgG, can deliver comparable therapeutic efficacy in uPA/SCID chimeric mice. Furthermore, those exogenous antibodies can't deliver durable therapeutic efficacy as repeated infusions are required to maintain therapeutic levels.

In one embodiment, the present invention emphasizes that the function of direct neutralization by HBV neutralizing antibodies that directly bind the attachment sites of full and subviral particles that prevent viral particles from attaching hepatocytes in human livers. Once there is no attachment to hepatocytes by viral particles and there will be no viral entry for initiating new rounds of infection. Thus, HBV antibodies against the attachment sites of viral particles provide most effective neutralization of HBV infectivity. Such antibody binding specificity is critical for blocking rcDNA and cccDNA replenishment.

In one embodiment, the present invention emphasizes that an increase in endogenous capacity to express HBV neutralizing antibody is the most effective in delivering durable cccDNA loss and elimination and HBV cure.

Several existing HBV therapeutic vaccines show disappointing results in clinical trials. The function of therapeutic vaccines relies on the host's immune cells from antigen processing and presentation, to signal transduction for activation of T and B cells to exert antiviral function. Furthermore, the number of specific T or B cells is limited and may not have enough capacity to produce high level of HBV neutralizing antibodies to block persistently high level of subviral particles (HBsAg) in HBV infected patients.

In the present invention, however, it is discovered that endogenous expression of HBV neutralizing antibodies should be independent of host's adaptive immune system and non-traditional antibody-expressing cells are required to expand endogenous capacity of expressing HBV neutralizing antibodies.

In one embodiment, this application invents AAV-anti-HBs vector as an HBV drug, which converts muscle cells to anti-HBs antibody producing cells after a single injection. Since this drug delivers endogenous expression of sustained high level of anti-HBs antibodies, the therapeutic efficacy is durable.

There is significant inefficiency in current mainstream HBV treatment that requires daily medication for lifetime because a treatment withdrawal frequently leads to HBV infection relapse and flares of liver injury that can result in fatal outcomes. In a continuous embodiment, the present invention shortens current infinite hepatitis B treatment duration to a definite treatment course that mainly requires a single injection.

In one embodiment, the present invention allows current HBV treatment to safely withdraw without HBV infection relapse and flares of liver injury

Unfortunately, in existing antiviral treatment methods, the achieved clearance of viral particle sis frequently reversed because the cells which were cleared of HBV infection are not protected from the attachment of virus particles and thus are subjected to frequent new rounds of infection.

In one embodiment, the present invention protects the cells that cleared HBV infection from recurrent HBV infection with sustained high level of HBV neutralizing antibodies. This makes the achieved clearance durable and progressively expanding until a complete HBV cure is established.

In one embodiment, the present invention shifts HBV treatment from currently inhibiting intracellular HBV replication to blocking new rounds of infection. In order to block new rounds of HBV infection, a sustained high level of anti-HBs antibody is required. There are a few approaches that can be explored to increase anti-HBs level. The first one is therapeutic vaccines. However, the performance of therapeutic vaccines is disappointing in clinical trials (Fontaine et al., Gut 64, 139-147 (2015).; Godon et al., Molecular therapy: the journal of the American Society of Gene Therapy 22, 675-684 (2014); Michel et al., Journal of hepatology 54, 1286-1296 (2011); Zoulim et al., Human vaccines & immunotherapeutics, (2019)). The second one is to repeatedly infuse exogeneous anti-HBs antibody, which are not durable (Galun et al., Hepatology 35, 673-679 (2002); Sneha V. Gupta ct al EASL 2021 International Liver Congress. (2021), pp. PO_43_ILC2021), or daily injection of entry inhibitor such as Myrcludex B (Bogomolov et al., Journal of hepatology 65, 490-498 (2016)), which does not react with viral particles and can't prevent HBV particles from attaching hepatocytes, resulting in poor efficacy in blocking new rounds of infection in established HBV infections in both HBV infected uPA/SCID chimeric mice and chronically infected humans. The inventor of the present invention chooses to utilize an engineered humoral immunology approach to develop a new HBV cure drug which can durable remedy anti-HBs deficiency in chronic HBV infected patients. Specifically, an optimized AAV vector is used to carry human anti-HBs genes, and the resultant drug is called AAV-anti-HBS vector, one of the leading candidates is HBVZ10 as described in the present application. HBVZ10 can endogenously express sustainably high level of anti-HBs antibody after a single injection into skeletal muscle cells. This engineered humoral immunology approach is adaptive immunity independent that bypasses the noted deficiency in both HBV specific T (Gehring and Protzer, Gastroenterology 156, 325-337 (2019)) and B cell (Burton et al., The Journal of Clinical Investigation, (2018)) in chronic HBV infection.

Table 3 summarizes major advantages of the present invention over current HBV treatment and drugs.

TABLE 3
Major advantages of the present invention
over existing HBV treatment and drugs

| | Present | Existing HBV |
|---|---|---|
| | Invention | treatment and drugs |
| Safely stop treatment | ✓ | X |
| One time expense | ✓ | X |
| Durable efficacy | ✓ | X |
| Anti-HBs conversion | ✓ | X |
| Blocking rcDNA replenishment | ✓ | X |
| Blocking cccDNA replenishment | ✓ | X |
| Depleting rcDNA | ✓ | X |
| Eliminating cccDNA | ✓ | X |
| HBV cure | ✓ | X |
| A single injection | ✓ | X |
| Independent host's adaptive immunity | ✓ | X |

Based on the discoveries discussed above, the inventor of the present application has developed some methods for effectively treating chronic hepatitis B infection.

In one embodiment, the present invention is directed to a method of treating human chronic hepatitis B virus (HBV) infection by reducing or eliminating cccDNA (covalently closed circular DNA) and/or rcDNA (relaxed circular DNA) in liver cell of an HBV infected patient for 3 months or longer.

In one embodiment, the HBV cccDNA level in the liver cell of the HBV infected patient is reduced to ≤1 copy/cell, ≤1 copy/10 cells, ≤1 copy/100 cells, ≤1 copy/1,000 cells, or ≤1 copy/10,000 cell.

In another embodiment, the level of rcDNA in the liver of the infected patient is reduced to ≤1 copy/cell, ≤1 copy/10 cells, ≤1 copy/100 cells, ≤1 copy/1,000 cells, or ≤1 copy/10,000 cell.

In another embodiment, the HBV cccDNA and rcDNA level in the liver cell of the HBV infected patient is durably reduced by administrating exogenous anti-HBs antibodies and/or viral or non-viral vectors or nano particles which endogenously express anti-HBs antibodies to maintain a high level of anti-HBs antibody in blood.

In another embodiment, the reduction or elimination of cccDNA in liver cell of the HBV infected patient is manifested by reducing or rendering undetectable the level of HBV DNA, HBeAg and HBsAg in the patient's blood by maintaining a high level of HBV neutralizing antibodies in the patient's blood.

In another embodiment, maintaining a high level of HBV neutralizing antibodies in patient's blood is represented by a complete sustained anti-HBs seroconversion, which is achieved by endogenously or exogenously infusing sufficient amount of high anti-HBs antibody to durably change serum HBsAg positive and anti-HBs negative (HBsAg+/anti-HBs−) to serum HBsAg negative and anti-HBs positive (HBsAg−/anti-HBS+).

In another embodiment, realizing a complete sustained anti-HBs seroconversion with sufficient anti-HBs antibody is one of the most effective methods to induce serum HBsAg loss or seroclearance.

In another embodiment, a complete sustained anti-HBs seroconversion is the primary goal of HBV curative treatment and is required for delivering more effective HBV functional cure.

In another embodiment, delivering more effective HBV functional cure requires 1^{st}clearing serum HBsAg or making serum HBsAg undetectable by expressing or administrating sustained high level of anti-HBs antibody

In another embodiment, a complete sustained anti-HBs seroconversion is required to establish a durable HBsAg seroclearance or functional cure among HBV drugs treated patients who transitorily cleared serum HBsAg or achieved functional cure by HBV drugs like nucleotide analogues, interferon, and/or NAP or RNAi.

In another embodiment, the high level of HBV neutralizing antibodies in the patient's blood is maintained for 1, 2, 3, 4, 5, 6, 7, or 8 months or longer.

In another embodiment, HBV neutralizing antibody is the antibody that can prevent viral particles from attaching hepatocytes, thus blocking HBV virions from entering hepatocytes effectively

In another embodiment, the high level of HBV neutralizing antibodies is at a level of >1, >10, >100, >200, or >300 μg/ml or >10, >100, >1,000, >10,000, >20,000 or >30,000 mIU/ml in the patient's blood.

In another embodiment, the HBV neutralizing antibodies is administered to the patient by infusing one, two or three exogenous human HBV neutralizing antibodies at a dose of about 0.1 mg/kg, 0.5 mg//kg, 1 mg/kg, 3 mg/kg, 5 mg/kg, 10 mg/kg, 20 mg/kg or ≥30 mg/kg body weight.

In another embodiment, the infusion of exogenous human neutralizing antibodies comprising a single, multiple or repeated infusions in order to maintain a level of antibody at >1, >10, >100, >200, or >300 μg/ml or >10,>100, >1,000, >10,000, >20,000 or >30,000 mIU/ml in the patient's blood for three months or longer.

In another embodiment, the HBV neutralizing antibodies is one, two or three endogenously expressed human neutralizing antibodies.

In another embodiment, the endogenous human HBV neutralizing antibodies are expressed by viral or non-viral vectors that include nanoparticles like lipid nanoparticles (LNP) or GalNAc particles.

In another embodiment, the viral or non-viral vectors contains DNA or mRNA sequences which encode the HBV neutralizing antibodies or antibody fragments. In one embodiment, the viral vectors are or comprise an adeno-associated virus (AAV) vector. In one embodiment, the AAV vectors comprise nucleic acid sequences encoding the HBV neutralizing antibodies or its antibody fragments and thus becomes AAV-anti-HBV vectors.

In one embodiment, the AAV-anti-HBV vectors are injected into muscle cells of HBV patients at a dose of about 1×10⁹ genome copies/kg, 1×10¹⁰ genome copies/kg, 1×10¹¹ genome copies/kg, 1×10¹² genome copies/kg, 2×10¹² genome copies/kg, 2.5×10¹² genome copies/kg or higher, respectively.

In one embodiment, the viral or non-viral vectors are injected to the patient at once or multiple times as needed.

In one embodiment, the depleting rcDNA through a combination of HBV neutralizing antibody with intracellular HBV replication inhibitors lead to a significant, nearly complete, or complete cccDNA loss.

In one embodiment, the intracellular HBV inhibitors comprise RT inhibitors, capsid inhibitors, RNAi drugs, nucleic acid polymers (NAP), interferons, innate immunity agonists and entry inhibitors.

In one embodiment, the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in chronic HBV infected people who are HBV treatment naïve.

In one embodiment, the AAV anti-HBV vectors expressing HBV neutralizing antibodies is used for blocking cccDNA, for inducing HBsAg seroclearance, for realizing a complete anti-HBs seroconversion, and for a more effective HBV cure replenishment for reducing and eliminating cccDNA, for inducing HBsAg seroclearance, for realizing a complete anti-HBS seroconversion, and for a more effective HBV cure in chronic HBV infected people who are receiving HBV treatment but can't safely withdraw antiviral drugs.

In one embodiment, the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in chronic HBV infected people who wish to become HBV infection free regardless of their infection status/phase or their treatment status (naïve or treated).

In one embodiment, the AAV anti-HBV vectors expressing HBV neutralizing antibodies is used for blocking cccDNA replenishment for reducing and eliminating cccDNA, for inducing

HBsAg seroclearance, for realizing a complete anti-HBs seroconversion, and for a more effective HBV cure in HBV infected pregnant women or patients with liver transplant.

In one embodiment, the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in people whose HBV infection has been clinically resolved but are at higher risk for HBV recurrence.

In one embodiment, the present invention is directed to a method of treating chronic HBV infection and/or providing protection against recurrence of HBV infection in a human patient, comprising administering to the HBV infected human patient a sufficient amount of HBV neutralizing antibodies or antibody fragments, wherein said amount of HBV neutralizing antibodies is at a level which blocks replenishing both rcDNA and cccDNA, enabling reducing and depleting rcDNA to <1 copy/cell and facilitating reducing and eliminating cccDNA <1 copy/cell.

In one embodiment, the treating of chronic HBV infection comprises treating newborns/children who have been infected by HBV. In another embodiment, the treating of chronic HBV infection comprises treating adults who have been infected by HBV.

In one embodiment, the HBV infected human patient is a chronic HBV infected individual who has been HBsAg positive for more than 6 months and have normal or elevated alanine aminotransferase (ALT) level. In another embodiment, the HBV infected human patient is an HBV positive pregnant woman, or an organ transplant recipient who is HBsAg positive or HBsAg negative/anti hepatitis B core antibody (anti-HBc) positive and is prone to recurrent HBV infection after transplant.

In one embodiment, the HBV neutralizing antibodies or antibody fragments are produced by HBV therapeutic vectors.

In one embodiment, the HBV therapeutic vectors comprise a mixed population of vectors each of which encodes one specific anti-HBs antibody or antibody fragment binding to one or more epitopes of HBV envelope proteins, or a single vector which encodes one HBV neutralizing antibody or antibody fragment binding to one or more epitopes of HBV envelope proteins. In one embodiment, the HBV neutralizing antibody or antibody fragment is one the antibodies described below.

### The Anti-Hepatitis B Drugs Used in This Invention

Any existing or newly developed anti-hepatitis B drugs which can eliminate or deplete cccDNA and/or rcDNA can be used in the methods of treatment described above. Such drugs include, but are not limited to, antibody drugs that are exogenous or endogenously expressed, small molecules drugs, peptide drugs and vector drugs. The following AAV-anti-HBs vectors-based HBV drugs that endogenously express anti-HBs antibodies after a single injection, were designed and tested in the present invention, and were found to be effective against chronic hepatitis infections when used in the treatment methods described above. A total of nine AAV-anti-HBS vectors comprise following sequences

Nucleic acid sequence ID NO: 1 encodes the amino acid sequence of sequence ID NO: 2. Sequence ID NO: 2 is the variable region of heavy chain of HBVZ10 human anti-HBs monoclonal IgG1 antibody against 4 serotypes of HBsAg.

Nucleic acid sequence ID NO: 3 encodes the amino acid sequence of sequence ID NO: 4. Sequence ID NO: 4 is the variable region of light chain of HBVZ10 human anti-HBs monoclonal IgGI antibody against 4 serotypes of HBsAg.

Nucleic acid sequence ID NO: 5 encodes the amino acid sequence of sequence ID NO: 6. Sequence ID NO: 6 is the variable region of heavy chain of HBVZ20 human anti-HBs monoclonal IgGI antibody against 4 serotypes of HBsAg.

Nucleic acid sequence ID NO: 7 encodes the amino acid sequence of sequence ID NO: 8. Sequence ID NO: 8 is the variable region of light chain of HBVZ20 human anti-HBs monoclonal IgG1 antibody against 4 serotypes of HBsAg.

Nucleic acid sequence ID NO: 9 encodes the amino acid sequence of sequence ID NO: 10. Sequence ID NO: 10 is the variable region of heavy chain of HBVZ30 human anti-HBS monoclonal IgGI antibody against 4 serotypes of HBsAg.

Nucleic acid sequence ID NO: 11 encodes the amino acid sequence of sequence ID NO: 12. Sequence ID NO: 12 is the variable region of light chain of HBVZ30 human anti-HBS monoclonal IgGI antibody against 4 serotypes of HBsAg.

Nucleic acid sequence ID NO: 13 encodes the amino acid sequence of sequence ID NO: 14. Sequence ID NO: 14 is the variable region of heavy chain of HBVZ40 human anti-HBS monoclonal IgGI antibody against 4 serotypes of HBsAg.

Nucleic acid sequence ID NO: 15 encodes the amino acid sequence of sequence ID NO: 16. Sequence ID NO: 16 is the variable region of light chain of HBVZ40 human anti-HBS monoclonal IgG1 antibody against 4 serotypes of HBsAg.

Nucleic acid sequence ID NO: 17 encodes the amino acid sequence of sequence ID NO: 18. Sequence ID NO: 18 is the variable region of heavy chain of HBVZ50 human anti-HBS monoclonal IgGI antibody against 4 serotypes of HBsAg.

Nucleic acid sequence ID NO: 19 encodes the amino acid sequence of sequence ID NO: 20. Sequence ID NO: 20 is the variable region of light chain of HBVZ50 human anti-HBS monoclonal IgGI antibody against 4 serotypes of HBsAg.

Nucleic acid sequence ID NO: 21 encodes the amino acid sequence of sequence ID NO: 22. Sequence ID NO: 22 is the variable region of heavy chain of HBVZ60 human anti-HBS monoclonal IgGI antibody against 4 serotypes of HBsAg.

Nucleic acid sequence ID NO: 23 encodes the amino acid sequence of sequence ID NO: 24. Sequence ID NO: 24 is the variable region of light chain of HBVZ60 human anti-HBs monoclonal IgG1 antibody against 4 serotypes of HBsAg.

Nucleic acid sequence ID NO: 25 encodes the amino acid sequence of sequence ID NO: 26. Sequence ID NO: 26 is the variable region of heavy chain of HBVZ70 human anti-HBS monoclonal IgGI antibody against 4 serotypes of HBsAg.

Nucleic acid sequence ID NO: 27 encodes the amino acid sequence of sequence ID NO: 28. Sequence ID NO: 28 is the variable region of light chain of HBVZ70 human anti-HBS monoclonal IgG1 antibody against 4 serotypes of HBsAg.

Nucleic acid sequence ID NO: 29 encodes the amino acid sequence of sequence ID NO: 30. Sequence ID NO: 30 is the variable region of heavy chain of HBVZ80 human anti-HBS monoclonal IgGI antibody against 4 serotypes of HBsAg.

Nucleic acid sequence ID NO: 31 encodes the amino acid sequence of sequence ID NO: 32. Sequence ID NO: 32 is the variable region of light chain of HBVZ80 human anti-HBs monoclonal IgG1 antibody against 4 serotypes of HBsAg.

Nucleic acid sequence ID NO: 33 encodes the amino acid sequence of sequence ID NO: 34. Sequence ID NO: 34 is the variable region of heavy chain of HBVZ90 human anti-HBS monoclonal IgGI antibody against 4 serotypes of HBsAg.

Nucleic acid sequence ID NO: 35 encodes the amino acid sequence of sequence ID NO: 36. Sequence ID NO: 36 is the variable region of light chain of HBVZ90 human anti-HBs monoclonal IgGI antibody against 4 serotypes of HBsAg.

Nucleic acid sequence ID NO: 37 is nucleic acid sequence of AAV vector which consists of 3758bp including two ITRs (inverted Terminal Repeat from AAV), chicken Beta-actin promoter, constant regions of human IgGI heavy and light chains, WPRE (woodchuck hepatitis virus posttranscriptional regulatory clement), and SV40 polyadenylation signal. This AAV vector (sequence ID NO: 37) allows cloning two variable regions of both heavy and light chains to express a full human IgGI monoclonal anti-HBs antibodies. In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 2 and an antibody VL, wherein the VL comprises the amino acid sequences of SEQ ID NO: 4, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 6 and an antibody VL, wherein the VL comprises the amino acid sequences of SEQ ID NO: 8, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 10 and an antibody VL, wherein the VL comprises the amino acid sequences of SEQ ID NO: 12, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 14 and an antibody VL, wherein the VL comprises the amino acid sequences of SEQ ID NO: 16, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 18 and an antibody VL, wherein the VL comprises the amino acid sequences of SEQ ID NO: 20, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 22 and an antibody VL, wherein the VL comprises the amino acid sequences of SEQ ID NO: 24, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 26 and an antibody VL, wherein the VL comprises the amino acid sequences of SEQ ID NO: 28, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 30 and an antibody VL, wherein the VL comprises the amino acid sequences of SEQ ID NO: 32, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to an isolated binding molecule or antigen-binding fragment thereof which specifically binds to HBV virions and/or HBsAg subviral particles comprising an antibody VH, wherein the VH comprises the amino acid sequences of SEQ ID NO: 34 and an antibody VL, wherein the VL comprises the amino acid sequences of SEQ ID NO: 36, or a variant thereof with at least 95% sequence homology. In one embodiment, the present invention is directed to a nucleic acid molecule encoding the above isolated binding molecule or antigen-binding fragment. In one embodiment, the present invention is directed to a vector comprising the above nucleic acid molecule.

In one embodiment, the present invention is directed to a composition comprising one or more of the isolated binding molecule or antigen-binding fragments described above.

In one embodiment, the present invention is directed to a pharmaceutical composition comprising one or more of the antibodies described above and a pharmaceutically acceptable carrier.

In one embodiment, a complete sustained anti-HBs seroconversion is the most effective way to induce serum HBsAg loss.

There are several drug candidates including RNAi drugs (siRNA or antisense RNA) and nucleic acid polymers (NAP) which directly suppress HBsAg synthesis or/and block HBsAg secretion from infected cells, leading to serum HBsAg reduction that takes months. The average serum HBsAg reduction by RNAi drug is about 1.5 log. Available data show that patients with high HBsAg level of >10,000 IU/ml respond poorly to the RNAi drugs. NAP-induced serum HBsAg reduction is usually accompanied by liver injury that raises safety concerns.

The present invention describes the most effective way to reduce serum HBsAg to undetectable level, that is through inducing a complete sustained anti-HBs seroconversion with providing sufficient amount of anti-HBs antibody either expressed or infused or a combination of both. As shown in FIG. 7, a complete anti-HBs seroconversion reduced serum HBsAg level from 3-5 logs depending on the baseline level, far more effective than average 1.5 log by RNAi drug. It takes only days to have serum HBsAg level undetectable if enough anti-HBs antibody that is >serum HBsAg level, is provided. Maintaining anti-HBs level>serum HBsAg level will make HBsAg loss durable.

In one embodiment, current lifetime daily medication can be shortened to a finite period as short as 9-week once added or combined with AAV-anti-HBs vectors or anti-HBs antibody.

The main reason for requiring lifetime daily medication is that the current therapy with nucleotide analogs does not address new rounds of infection occurring in chronic HBV infection, nor improve insufficient anti-HBs level, leading to requiring lifetime treatment to suppress viral replication that will rebound if the therapy is stopped. As demonstrated in FIG. 7, a late addition of AAV-anti-HBs vector and exogenous anti-HBS antibody to 9-week entecavir treatment achieved HBV functional cure in 5 of 8 the mice with a complete anti-HBs seroconversion and established proof of concept that currently lifetime HBV treatment can be shortened to a finite period as short as 9-week. Thus, the key method to shorten HBV treatment is to add enough AAV-anti-HBs vectors and/or exogenous anti-HBs antibody to current therapies, and anti-HBs antibody level should be constantly greater than serum HBsAg level.

In one embodiment, non-durable serum HBsAg loss or functional cure mediated by current therapies or by spontaneous HBV clearance can be prevented and converted to durable HBsAg loss and functional cure.

Serum HBsAg loss or HBV functional cure can be achieved spontaneously or through current therapies, but such cure or HBsAg loss is rare and can be non-durable. The main reason for non-durable HBsAg loss or functional cure is the absence of sufficient anti-HBs antibody in chronic HBV infected patients. As demonstrated in FIG. 7, serum HBsAg loss or HBV functional cure became durable if sufficiently high level of anti-HBs antibody were provided and maintained. Thus, the key method to achieve a durable serum HBsAg loss and/or durable HBV functional cure is to provide and maintain anti-HBs level that should be constantly greater than serum HBsAg level.

In one embodiment, the present invention is directed to a method of treating chronic HBV infection and providing protection against recurrence new rounds of HBV infection in a human patient, comprising administering to the HBV infected human patient a sufficient amount of HBV neutralizing antibodies or antibody fragments, wherein said amount of HBV neutralizing antibodies is at a level which blocks replenishing both rcDNA and cccDNA, enabling reducing and depleting rcDNA to <1 copy/cell and facilitating reducing and eliminating cccDNA <1 copy/cell, inducing HBsAg seroclearance, realizing a complete anti-HBs seroconversion, and delivering a more effective HBV cure.

In one embodiment, the treating of chronic HBV infection comprises treating newborns/children who have been infected by HBV.

In one embodiment, the treating of chronic HBV infection comprises treating adults who have been infected by HBV.

In one embodiment, the HBV infected human patient is a chronic HBV infected individual who has been HBsAg positive for more than 6 months and have normal or elevated alanine aminotransferase (ALT) level.

In one embodiment, the HBV infected human patient is an HBV positive pregnant woman, or an organ transplant recipient who is HBsAg positive or HBsAg negative/anti hepatitis B core antibody (anti-HBc) positive, andpositive and is prone to recurrent HBV infection after transplant.

In one embodiment, the HBV neutralizing antibodies or antibody fragments are produced by HBV therapeutic vectors.

In one embodiment, the HBV therapeutic vectors comprise a mixed population of vectors each of which encodes one specific anti-HBs antibody or antibody fragment binding to one or more epitopes of HBV envelope proteins, or a single vector which encodes one HBV neutralizing antibody or antibody fragment binding to one or more epitopes of HBV envelope proteins.

### EXAMPLES

### Example 1. Experimental Procedures Using uPA/SCID Chimeric Mice for HBV Infection and Treatment

A total of 32 male uPA/SCID chimeric mice were infected with an inoculum of 5×10⁸ HBV DNA copies on day 1. AAV vectors that express malaria antibody (antibody specificity control) or anti-HBs antibodies, were intramuscularly injected into right thigh muscle cells at a dose of 1×10¹¹ genomic copies 7-week post infection (pi), then monitored for additional 30-week (FIG. 7). There were 20 animals remaining until day 183 and 10 remaining at day 253 (termination day).

Serologic data were reported until day 183 for the reason to have enough number of animals for statistical analysis. Intrahepatic HBV DNA data from 18 livers, 8 of which collected around day 204 and the remaining 10 collected at day 253. Each liver is randomly sampled for at least 20 times by cutting 20-30 mg tissue for each sampling, were also analyzed. HBV level in each sampling is individually quantitatively determined and average HBV level from 20 samplings of each liver was calculated.

### Example 2. Kinetic Serum Antibody Level in two Groups

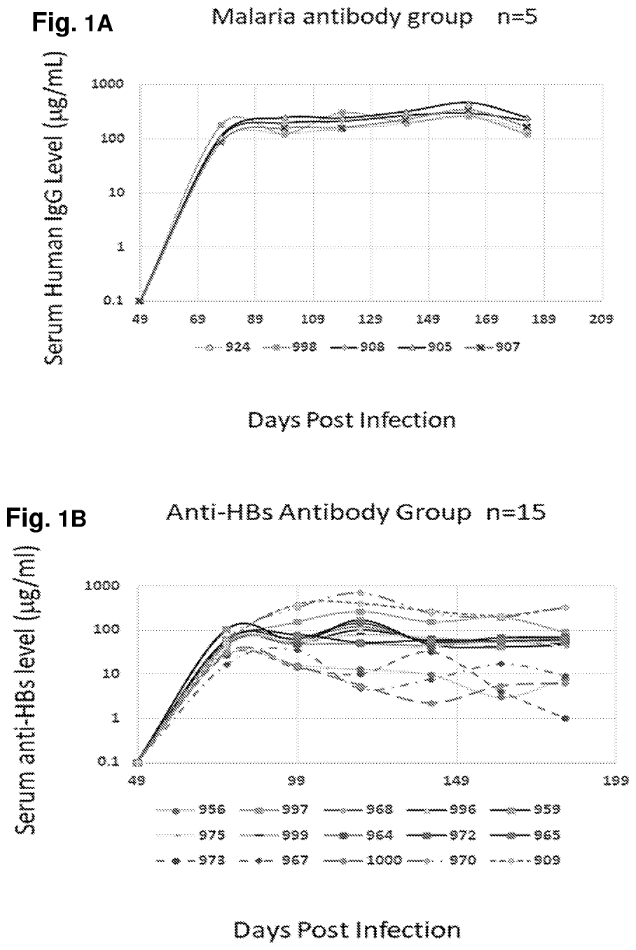

As shown in FIG. 1A, 5 mice were injected with AAV vector expressing malaria antibody as expression control at day 49 pi, the expressed antibody level was sustained above 100 μg/ml throughout the observation period (around 200 days, timepoint after day 183 not shown), suggesting this optimized AAV vector can express sustained high level of antibody after a single injection.

Among the 15 animals with anti-HBs treatment (FIG. 1B), 13 were injected with AAV-anti-HBs vectors expressing anti-HBs antibodies, and the remaining two animals No. 970 and No. 909 were triweekly injected with exogenous mouse anti-HBs antibody at a dose of 250 μg per injection started at day 74 pi as control. Kinetic anti-HBs level in most mice was around 100 μg/ml, comparable to malaria antibody level, but also detected around 10 μg/ml or lower in a few animals. Example 3. HBV infection level was significantly lower in animals treated with anti-HBS antibodies

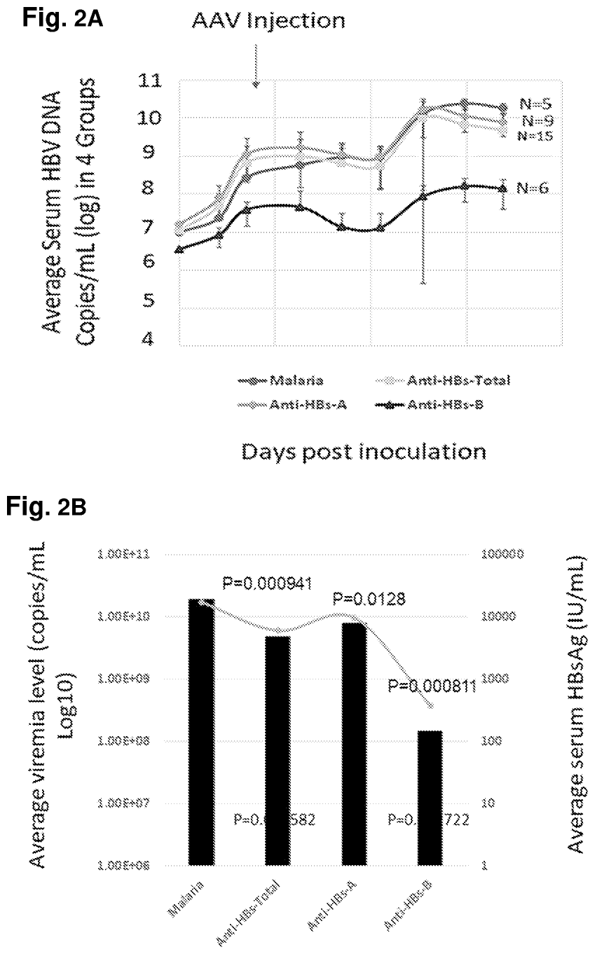

The average viremia among all animals ranged between 1E8 and 1E9 HBV DNA copies/ml at day 49 pi, suggesting all infectible cells must have been infected because their livers had been exposed to viremia of >1×10⁷ HBV DNA copies/ml for 35-day (from day 14 to day 49 pi, FIG. 2A) since it only takes <24 hours for a Hepadnavirus to establish in vivo or in vitro infection in a susceptible cell. Average viremia in 15 mice treated with anti-HBs antibody (anti-HBs-Total) was lower than that in malaria antibody group before AAV injection at day 49. Viremia in anti-HBS-Total group experienced decline and became lower than malaria antibody group after AAV vector or anti-HBs injection, demonstrating a response to anti-HBs treatment. Subsequently, viremia in both groups experienced a surge and reached the peak. Reduced viremia in anti-HBs-Total group became detectable again after the peak while viremia in malaria group remained steady (FIG. 2A).

The 15 mice in anti-HBs group were further divided into anti-HBS-A (n=9) and anti-HBS-B group (n=6) based on if HBV infection reached a typical peak viremia (FIG. 2A). Viremia in anti-HBs-A group reached a typical peak (serum HBV DNA level >1E¹⁰ copies/ml), then followed by viremia reduction, and HBV infection in anti-HBS-B group was delayed, not yet reached the peak. The difference in viremia between malaria antibody and anti-HBs-B groups was >100-fold over several timepoints.

Average viremia at day 183 was significantly lower in both anti-HBs-Total and anti-HBS-B group compared to malaria group (p=0.029 and p=0.012, see FIG. 2B). Average serum HBsAg level at day 183 in all 3 anti-HBs groups was significantly lower when compared to malaria group (FIG. 2B. p=0.0009 for anti-HBs-Total, p=0.0128 for anti-HBS-A and p=0.0008 for anti-HBs-B).

Both viremia and HBsAg data suggest that blocking new rounds of infection after all infectible cells infected can significantly lower HBV infection level. In other words, HBV infection level is determined by the level of new rounds of infection.

### Example 4. HBV Functional Cure in Test Animal No. 970

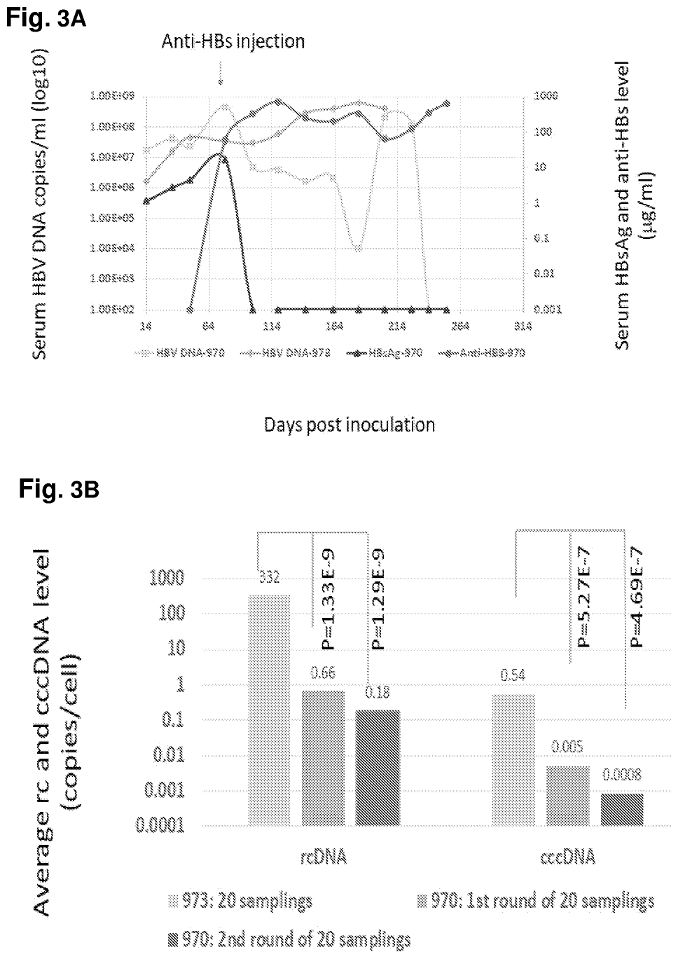

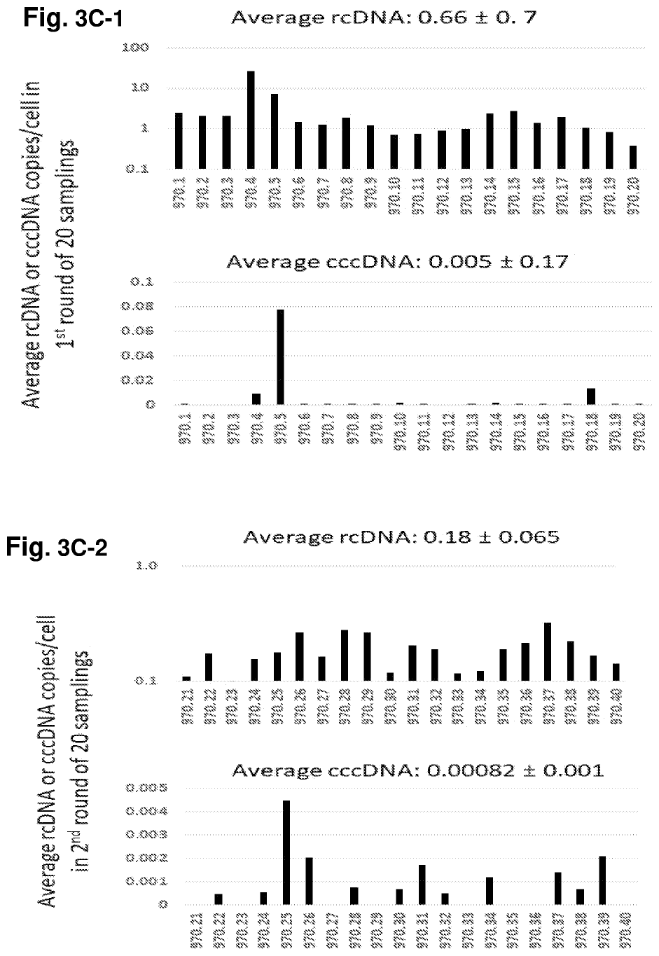

Baseline viremia in test animal No. 970 reached 4.6×10⁸ HBV DNA copies/ml before anti-HBs treatment, resembling the high-end viremia in CHB patients. Viremia was reduced by 4-log from 1×10⁸ to 1×10⁴ with anti-HBs treatment, it was briefly surged to the pre-treatment level, then became undetectable for 4 weeks until termination (FIG. 3A).

Serum HBsAg in animal No. 970 became undetectable following anti-HBs treatment for consecutive 6 months while anti-HBs level reached 60ug/ml at day 77 pi and sustained at >100 μg/ml in most timepoints of the observation period (FIG. 3A), that represented a complete sustained anti-HBs seroconversion.

To provide a reference to pre-treatment level of intrahepatic rcDNA and cccDNA in FIG. 3B, we used a test animal No 973 who had comparable baseline viremia to pre-treatment level of test animal No. 970 as reference plotted in FIG. 3A.

Serologic data suggests that a functional cure of HBV infection was achieved in test animal No. 970. This HBV curing course took about 6 months (from day 74 with the 1st anti-HBs injection to day 239 with undetectable HBV DNA).

To our knowledge, test animal No. 970 represents the 1st case of successful HBV functional cure with a tested intervention in HBV infected chimeric mouse model that supports a robust HBV infection.

Average intracellular rcDNA level was 0.66 and 0.18 copies/cell in the 1st and 2nd round of 20 liver samplings in test animal No. 970, which were 500 and 1800folds lower than animal 973 (p=1.33×10⁻⁹ and 1.29×10⁻⁹) whose liver serves as pre-treatment level control. Average cccDNA level was 1 copy per 200 cells (0.005 copy/cell) and 1 copy per 1250 cells (0.0008/cell) in the two rounds of 20 samplings in animal 970, respectively, i.e., cccDNA was lower by between 100 and 675 folds compared to test animal No. 973 (p=5.27×10⁻⁷ and 4.69×10⁻⁷, FIG. 3B). The results show that both rcDNA and cccDNA underwent significant losses in test animal No. 970.

Average rcDNA level in 7 of 20 samplings of the first round and 20 of 20 samplings of the second round was ≤1 copies/cell and no cccDNA was detected in 3 and 8 cccDNA samplings of the 1st and 2nd rounds of 20 samplings, respectively, while the cccDNA levels detected in most samplings were very low from 1 copy per 77 cells to 11,100 cells (FIGS. 3C1 and C2), suggesting an overwhelming majority of infected cells had lost cccDNA in this liver on day 253.

Since anti-HBs antibody mainly functions extracellularly and cccDNA elimination was a result of spontaneous cccDNA loss. The cccDNA data from test animal No. 970 also suggests that anti-HBs monotherapy can potentially lead to a complete cccDNA clearance through blocking rcDNA replenishment with sustained high level of anti-HBs antibody.

### Example 5. Dynamic cccDNA Status and Spontaneous cccDNA Loss

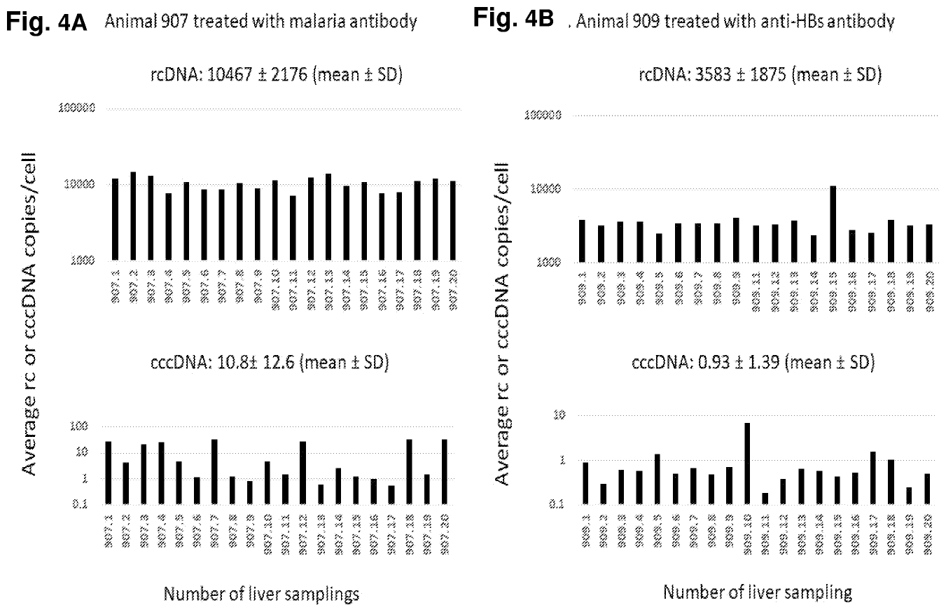

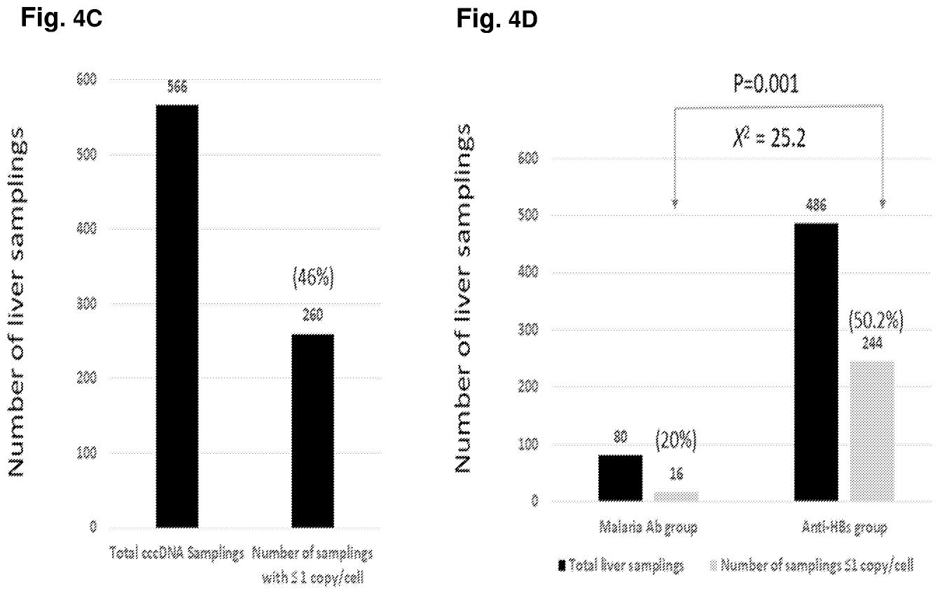

Diverse cccDNA levels were observed in different samplings of the same liver, for instance, the lowest cccDNA level was 0.5 copies/cell (sampling 17) while the highest cccDNA level was 31 copies/cell (sampling 18), and the difference was 60-fold among samplings in animal 907 treated with malaria antibody (FIG. 4A). In test animal No. 909 which was treated with anti-HBS antibody, the highest level was 6.6 copies/cell and the lowest was 0.18 copies/cell (FIG. 4B) and the difference was >30-fold. The diverse cccDNA levels in both test animal No. 909 and test animal No. 907 demonstrate dynamic cccDNA status, i.e., cccDNA was amplified in some cells while lost in the other cells. It is noted that the average rcDNA levels among 20 samplings of test animal No. 907 and test animal No. 909 were 10,467 (ranged between 6,992 and 14,610 copies/cell) and 3,583 copies/cell (ranged between 2,304 and 11,145 copies/cell), respectively, suggesting that most cells among samplings were HBV infected cells.

It was also observed in this experiment that cccDNA molecules were already lost in a fraction of infected cells.

An average cccDNA level of ≤1 copy/cell was detected in 5 of 20 samplings in test animal No. 907 treated with malaria antibody and detected in 17 of 20 samplings in test animal No. 909 treated with anti-HBs antibody (FIGS. 4A and B). A total of 566 cccDNA samples have been analyzed, cccDNA level ≤1 copy/cell was detected in 260 (46%) of them (FIG. 4C). When average cccDNA level is ≤1 copy/cell, cccDNA in some cells is >1 copies while absent in other cells, suggesting that cccDNA was already lost in a fraction of infected cells in HBV infected human liver cells in chimeric mice. It is noted that all HBV infectible cells must have been infected in the human liver cells by day 253 pi when livers were harvested.

The percentage of liver samplings with average cccDNA level ≤1 copy/cell is significantly higher in anti-HBs group than malaria antibody group (p<0.001, FIG. 4D), suggesting that blocking new rounds of infection likely blocks de novo infection mediated cccDNA replenishment in cells that spontaneously lost cccDNA, expanding the number of cells with cccDNA ≤1 copy/cell, i.e., blocking new rounds of infection establishes and expands a net cccDNA loss, which could be replenished in the absence of sufficient anti-HBs antibody.

The observed spontaneous cccDNA loss in a portion of infected cells is an important finding not only for understanding cccDNA biology, but also for HBV cure treatment, providing evidence that cccDNA can be spontaneously eliminated from infected cells. This forms a foundation for establishing HBV cure.

### Example 6. rcDNA Level Significantly Lowered by Blocking new Rounds of Infection

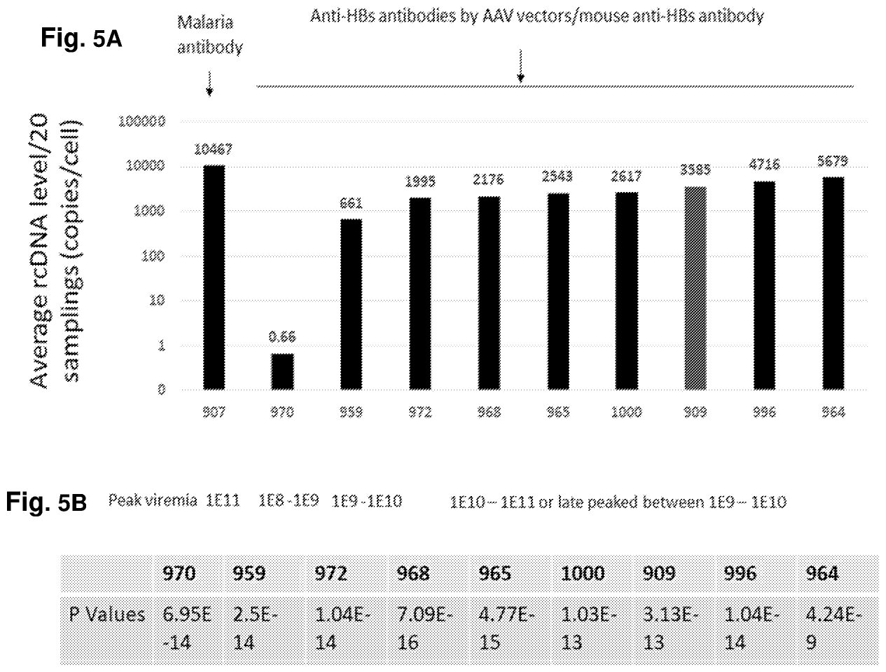

The intrahepatic rcDNA levels of from HBV infected uPA/SCID chimeric mice were analyzed in two sets of liver samples. The 1st set of sample includes eight livers, three of them were treated with AAV vector-expressing malaria antibody as a treatment control, and the remaining five livers were treated with anti-HBs antibodies, including four with AAV vectors expressing anti-HBs antibodies and one injected with mouse anti-HBs antibody. Seven of the eight livers were collected around day 204 pi and one (animal No. 969) was collected on day 183. Treatment with the expressed or injected anti-HBs antibody lowered viremia from two-fold to 100-fold (FIG. 5A). The average rcDNA level from 20 samples of each of the three livers with malaria antibody treatment ranged from nearly 2,000 to 2,700 copies/cell, and an average of 219 to 454 copies/cell was detected in the five livers treated with anti-HBs antibodies, that is, rcDNA level was significantly lowered by at least 1500 copies/cell during the 130-day anti-HBs treatment (see FIG. 5B). The P-values ranged between E-23 and E-25.

The 2^{nd} set of livers were collected on day 253 and includes 10 livers. One liver was treated with AAV vector-expressing malaria antibody and the other nine livers were treated with anti-HBS antibodies (seven with the expressed anti-HBs antibodies by AAV-anti-HBs vectors and the other two livers of animals No. 970 and 909 with injected mouse anti-HBs antibody). The average rcDNA level from 20 samples in animal No. 907 treated with malaria antibody was 10,467 copies/cell, and the average rcDNA level was significantly lower across all nine livers treated with anti-HBs antibodies (see FIG. 5C). The efficiency to lower rcDNA levels was largely peak viremia level dependent, assuming comparable virion secretion rates among different animals. For instance, rcDNA level in animal 970 with viremia between 1×10⁸ and 1×10⁹ HBV DNA copies/ml was 0.66 copies/cell and in animal 959 with viremia between 1×10⁹ and 1×10¹⁰ HBV DNA copies/ml, was 661 copies/cell, a reduction of nearly 10,000 copies/cell compared to animal 907. The average rcDNA level was lowered by 4,000 to 8,000 copies/cell in the remaining seven livers with viremia between 1×10¹⁰ and 1×10¹¹ or late peaked at >1×10⁹ HBV DNA copies/ml. The P-values ranged between E-9 and E-16 (see FIG. 5D).

### Example 7. Add-on of AAV-anti-HBs Vectors to 9-Week Entecavir (ETV) Treatment

In another round of experiment, HBV-infected uPA/SCID chimeric mice were divided into 3 groups: i) Untreated. ii) ETV monotherapy, and iii) ETV with add-on of AAV-anti-HBs vectors. ETV treatment started week 6 pi (post infection) through intraperitoneal injection at a dose of 0.3 mg/kg, three times a week for 9 weeks. In the add-on group, AAV-anti-HBs vectors that express human monoclonal anti-HBs antibodies were administrated through intramuscular injection at a dose of 1E11 genomic copies at week 6 of ETV treatment or week 11 pi. ETV was withdrawn at week 14 pi and HBV infection was monitored until week 30.

### HBV Infection in Untreated Mice

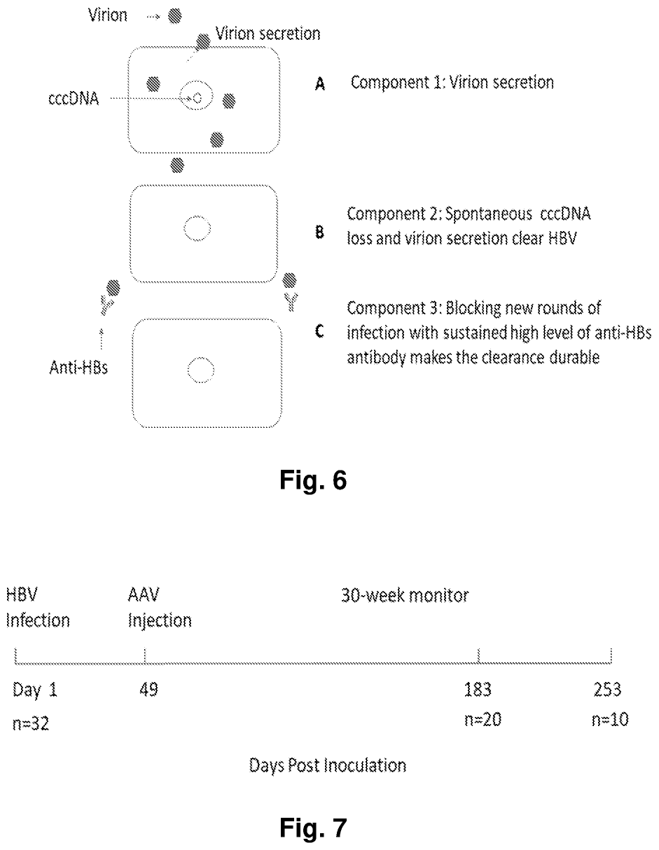

HBV infection reached the peak level around day 81 pi, demonstrated by viremia ranged between 3E9 and 2E10 HBV DNA copies/ml and by serum HBsAg ranged between 5,000 and 10,000 IU/ml. Serum HBV DNA and HBsAg were sustained at steady levels thereafter (see FIGS. 6A and B).

### HBV Infection Relapsed After ETV Cessation in ETV Monotherapy Group

Baseline viremia at day 35 (treatment started) ranged between 8E7 and 1E9 in this group. As expected, ETV treatment reduced viremia by 2-3 logs while no impact on serum HBsAg level was detected. After ETV withdrawal, HBV DNA level gradually bounced back and eventually reached a level comparable to the untreated animals while serum HBsAg level stayed steady (FIGS. 6A and B).

HBV relapse was prevented in animals treated with ETV and add-on of AAV-anti-HBS vectors.

Baseline viremia at day 35 ranged between 3E7 and 1E9 in this group. Serum HBV DNA level was 2-3 logs reduced to 1E6 or lower during ETV treatment. No HBV relapse in both serum HBV DNA and HBsAg levels was observed in all 10 animals with add-on therapy throughout the observation periods after ETV withdrawal (FIGS. 6C and D). The successful prevention of HBV relapse with anti-HBs antibody monotherapy confirmed that HBV relapse is mainly caused by new rounds of infection.

The successful prevention of HBV relapse reflects the lack of expansion of HBV infection following ETV withdrawal. It also indicates no significant HBV reduction either, i.e. HBV infection was maintained at an equilibrium status featuring dual HBsAg and anti-HBs positivity. Such a co-existence serologic profile signaled relatively insufficient anti-HBs level that couldn't completely bind all serum HBsAg articles, or new rounds of infection were not completely blocked. A complete anti-HBs antibody seroconversion is required for HBV functional cure

To test if boosting anti-HBs level would change therapeutic outcomes, 8 of the 10 mice received triweekly injection of 250 μg of exogenous mouse anti-HBs antibody, which started at different timepoints (6 of them shown in FIGS. 7A-F). As a result of boosting anti-HBs level, serum HBsAg loss and a complete anti-HBs seroconversion were established in all 8 mice (FIG. 6C). Furthermore, HBV functional cure was achieved in 5 of the 8 mice by the end of experiment (FIGS. 7A-E).

This experiment shows that different anti-HBs levels determine different outcomes: 1. The absence of anti-HBs caused HBV relapse that was featured with only HBsAg positivity. 2.Suboptimal anti-HBs level, i.e. anti-HBs level ≤serum HBsAg level that was featured with dual positivity of HBsAg and anti-HBs, may suppress significant HBV relapse but can't completely block new rounds of infection, resulting in a steady infection level. 3. Sufficient anti-HBs level, i.e., anti-HBs level>serum HBsAg level that resulted in HBsAg loss and a complete anti-HBS seroconversion that may have completely blocked new rounds of infection, leading to subsequent functional cure featured with HBsAg−/anti-HBS+. All HBV functional cures in the 6 mice occurred upon or after a complete anti-HBs seroconversion (FIG. 3 and FIGS. 7A to 7E). HBV functional cure had yet to occur in the remaining 3 mice after anti-HBs seroconversion because of insufficient observation duration: 2 died one or 3 weeks after anti-HBs seroconversion and the last one 971 had both high baseline viremia 1E9 copies/ml and serum HBsAg level 11,000 IU/ml, and achieved anti-HBs seroconversion at day 165 but remained HBV DNA positive until termination (day 218, FIG. 7F).

Example 8. A complete anti-HBs seroconversion delivers more effective HBV functional cure that features higher rate of HBV cure achieved in a period of months compared to the rarity of spontaneous clearance or current therapies-mediated HBV cure that usually takes years or decades.

As shown in FIG. 7, HBV functional cure achieved in 4 of 5 HBV infected chimeric mice with a complete anti-HBs seroconversion during 16-week of observation. HBV cure had yet to occur in the last animal that had the highest baseline HBsAg and HBV DNA level and achieved a complete anti-HBs seroconversion at day 162. However, this animal was only followed for 56 days before the experiment was terminated on day 218.

Such results are consistent with the observation that anti-HBs antibody seroconversion marks the resolution of acute HBV infection²¹. In fact, a complete anti-HBs seroconversion indicates that anti-HBs level already exceeds serum HBsAg level that leads to a completely blocking new rounds of infection and establishing progressive HBV clearance. Thus, the primary goal in HBV cure treatment must be to establish a complete anti-HBs seroconversion with sufficient anti-HBs antibody as early as possible.

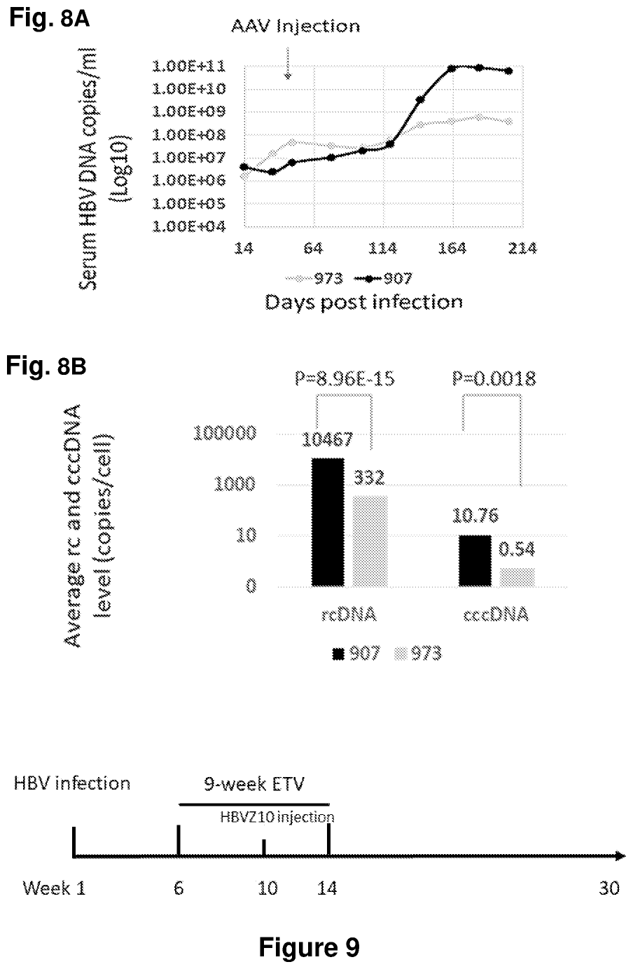

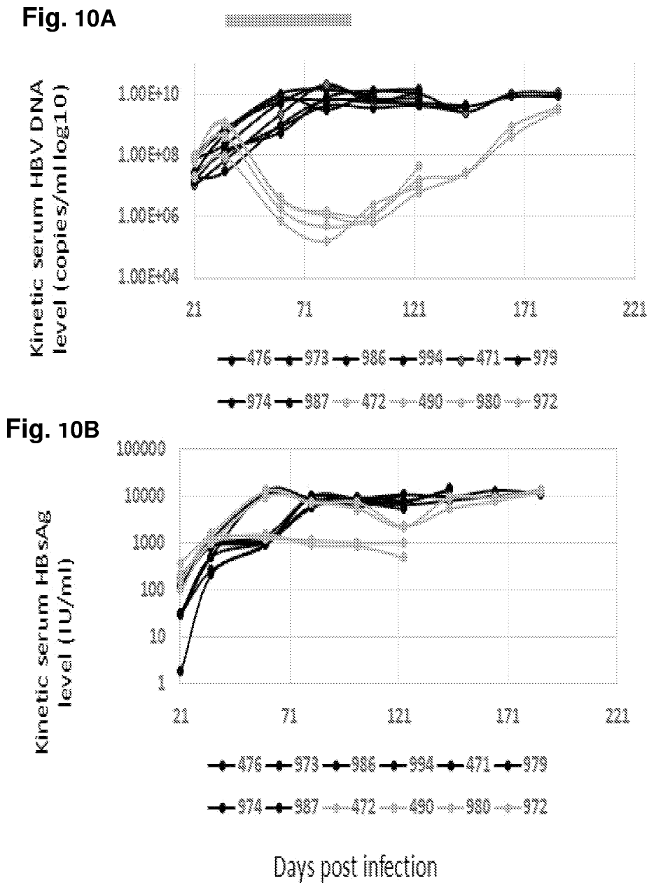

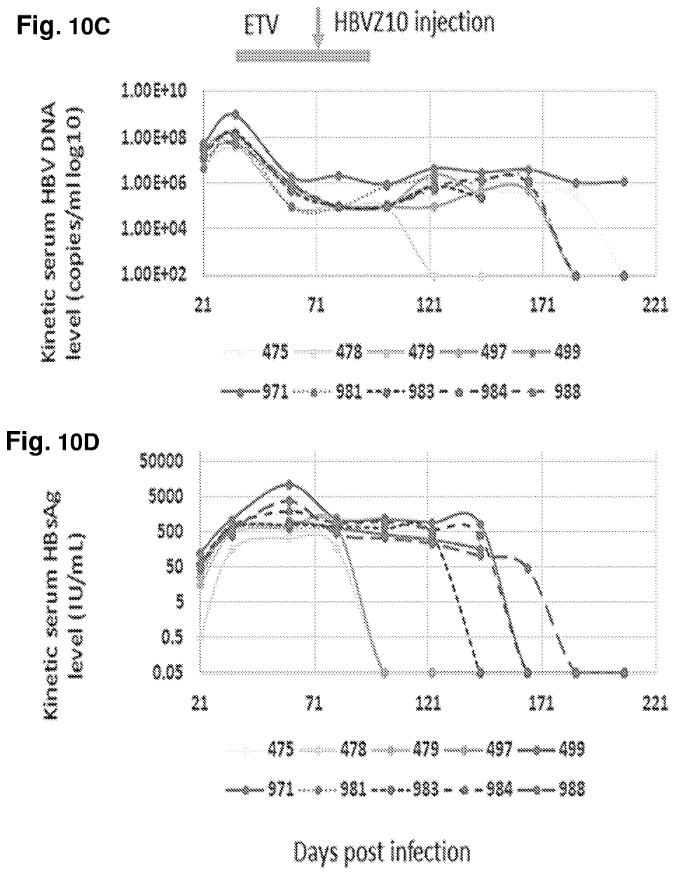

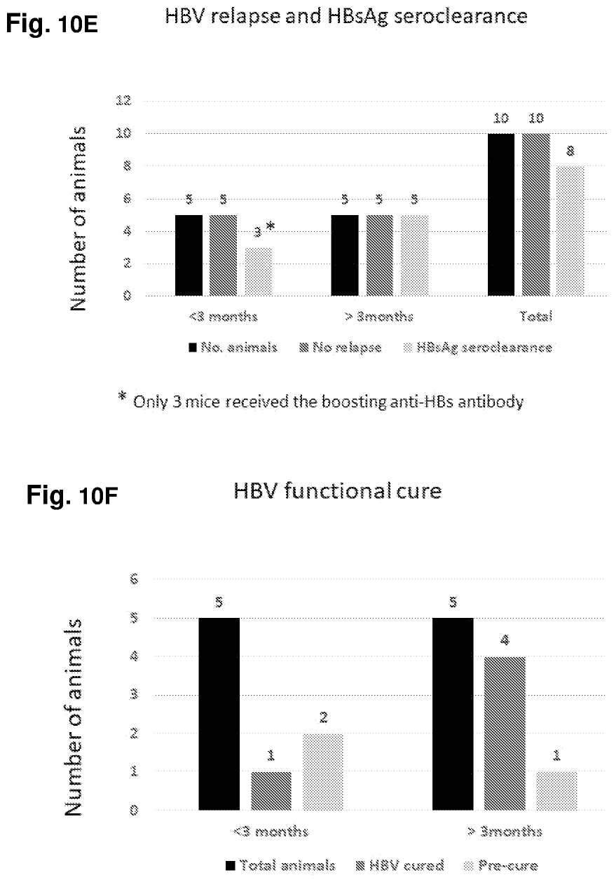

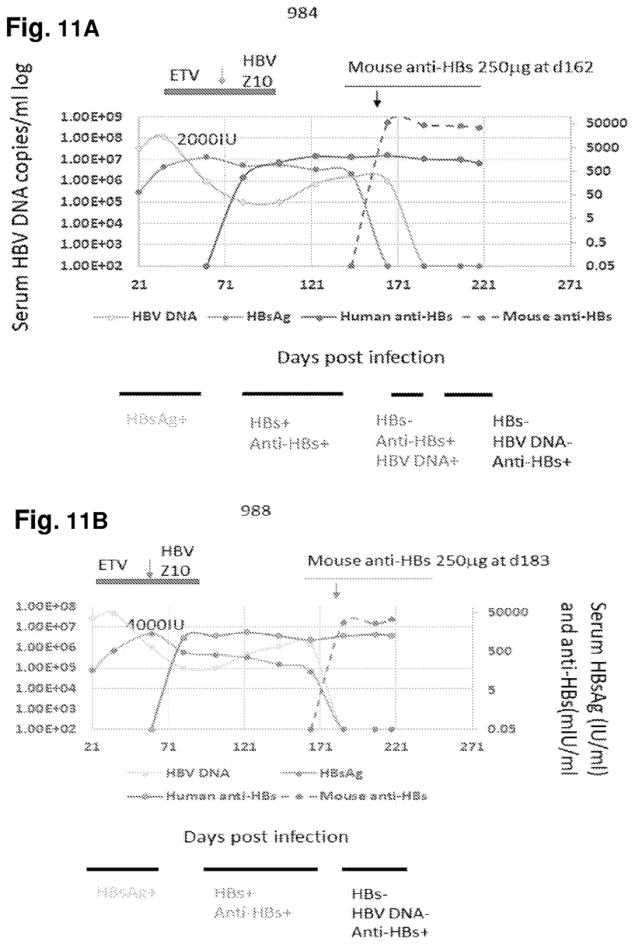

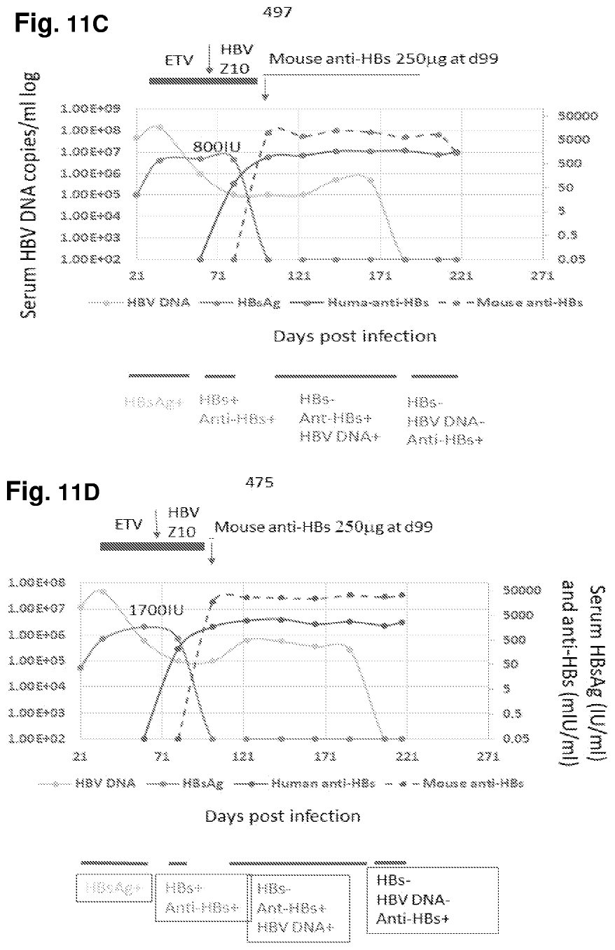

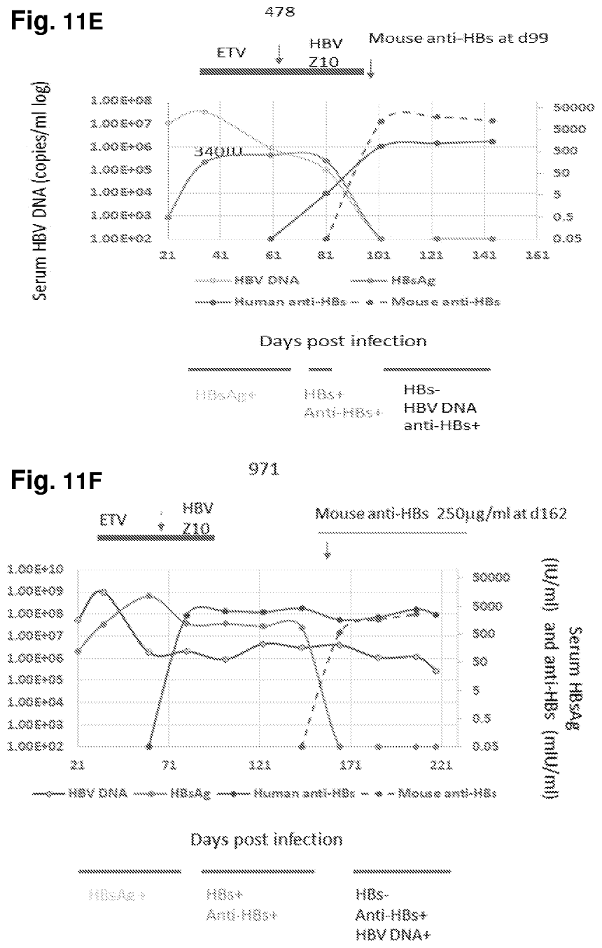

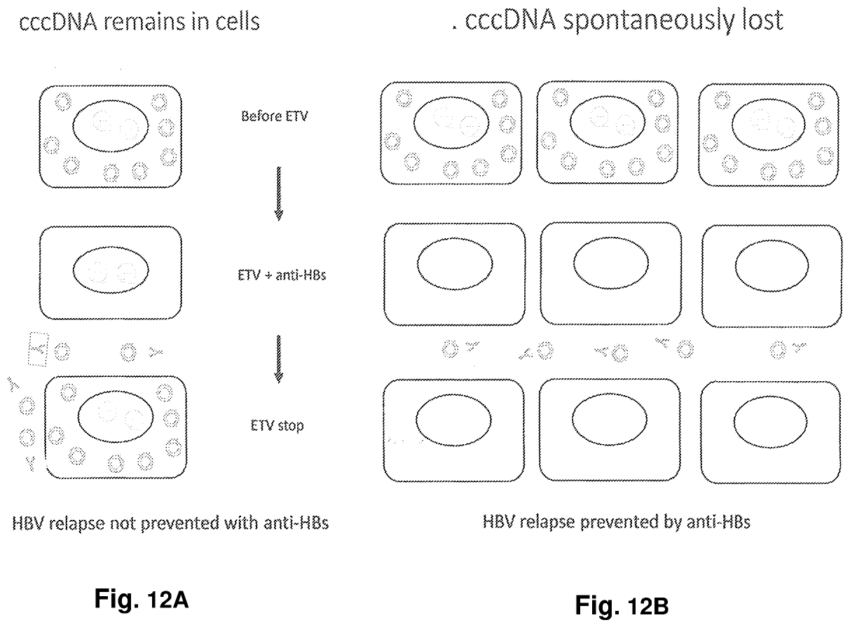

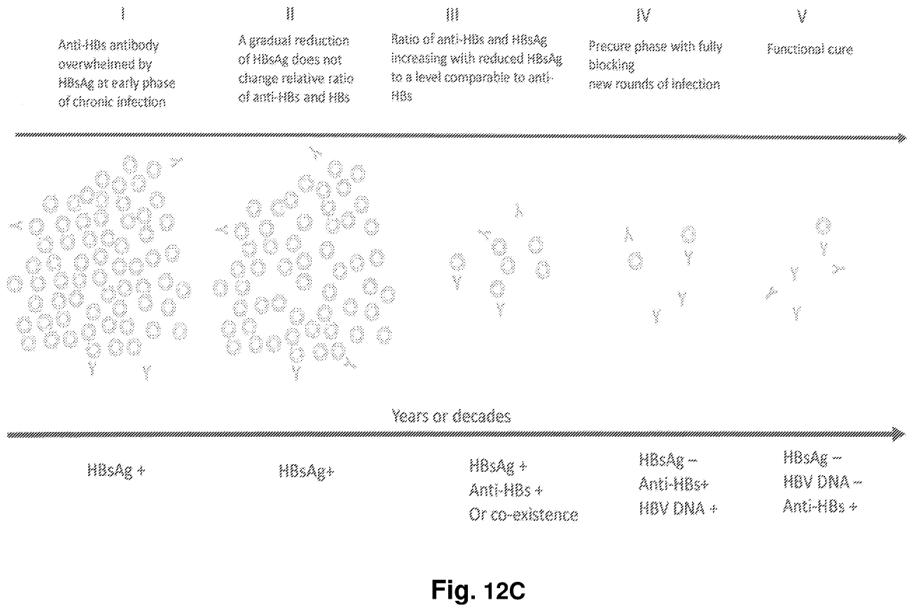

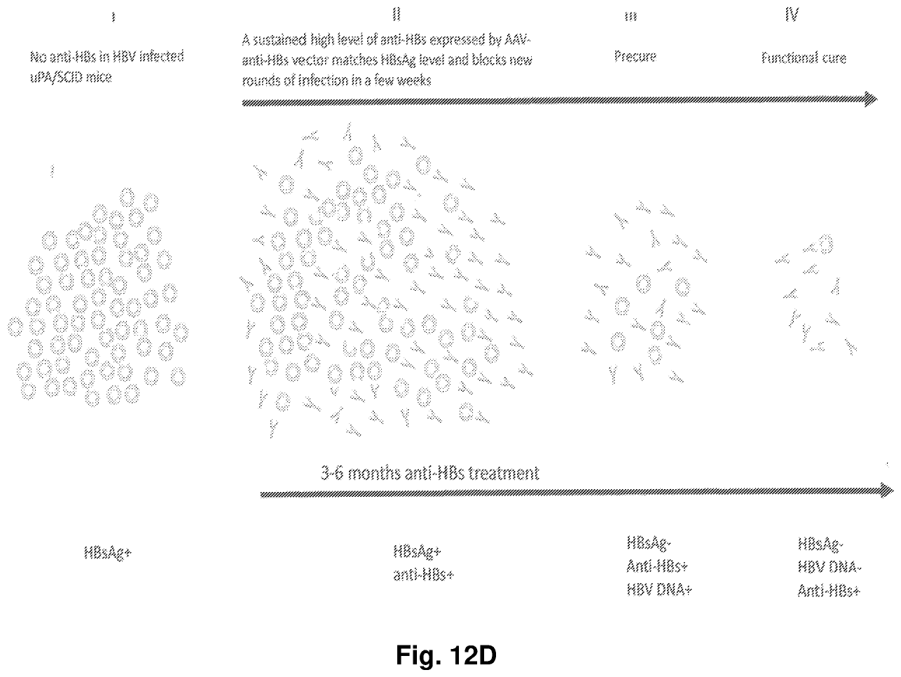

Spontaneous HBV cure occurs in chronic HBV infection and is mainly driven by gradual reduction of HBsAg level that can't immediately achieve a complete anti-HBs seroconversion, leaving new rounds of infection ongoing. This is why it takes years/decades to achieve spontaneous HBV functional cure (FIG. 8C). However, a complete anti-HBs seroconversion can be quickly realized upon expressing sustained and high level of anti-HBs antibody, which significantly reduces HBV curing duration (FIG. 8D) compared to spontaneous HBV cure course.

1. A method of treating human chronic hepatitis B virus (HBV) infection by reducing or eliminating cccDNA (covalently closed circular DNA) and/or rcDNA (relaxed circular DNA) in liver cell of an HBV infected patient for 3 months or longer.

2. The method of claim 1, wherein the HBV cccDNA level in the liver cell of the HBV infected patient is reduced to <1 copy/cell, <1 copy/10 cells, <1 copy/100 cells, <1 copy/1,000 cells, <1 copy/10,000 cell, or not detectable.

3. The method of claim 1, wherein the level of rcDNA in the liver of the infected patient is reduced to <1 copy/cell.

4. The method of claim 1, wherein the HBV cccDNA and rcDNA level in the liver cell of the HBV infected patient is durably reduced by administrating exogenous anti-HBs antibodies and/or viral or non-viral vectors or nano particles which endogenously express anti-HBs antibodies to maintain a high level of anti-HBs antibody in blood.

5. The method of claim 4, wherein the reduction or elimination of cccDNA in liver cell of the HBV infected patient is manifested by reducing or rendering undetectable the level of HBV DNA, HBeAg and HBsAg in the patient's blood by maintaining a high level of HBV neutralizing antibodies in the patient's blood.

6. The method of claim 5, wherein the maintaining a high level of HBV neutralizing antibodies in patient's blood is represented by a complete sustained anti-HBs seroconversion, which is achieved by endogenously expressing or exogenously infusing sufficient amount of anti-HBs antibody to durably change serum HBsAg positive and anti-HBs negative (HBsAg+/anti-HBs−) to serum HBsAg negative and anti-HBs positive (HBsAg−/anti-HBS+).

7. The method of claim 6, wherein realizing a complete sustained anti-HBS seroconversion with sufficient anti-HBs antibody is one of the most effective methods to induce serum HBsAg loss or seroclearance.

8. The method of claim 6, wherein a complete sustained anti-HBS seroconversion is the primary goal of HBV curative treatment and is required for delivering more effective HBV functional cure.

9. The method of claim 8, wherein delivering more effective HBV functional cure requires 1^{st} clearing serum HBsAg or making serum HBsAg undetectable by expressing or administrating sustained high level of anti-HBs antibody.

10. The method of claim 8, wherein a complete sustained anti-HBS seroconversion is required to establish a durable HBsAg seroclearance or functional cure among HBV drugs treated patients who transitorily cleared serum HBsAg or achieved functional cure by HBV drugs like nucleotide analogues, interferon, and/or NAP or RNAi.

11. The method of claim 5 wherein high level of HBV neutralizing antibodies in the patient's blood is maintained for month or longer.

12. The method of claim 10, wherein HBV neutralizing antibody is the antibody that can prevent viral particles from attaching hepatocytes, thus blocking HBV virions from entering hepatocytes effectively.

13. The method of claim 11, wherein the high level of HBV neutralizing antibodies is at a level of >1 mIU/ml in the patient's blood.

14. The method of claim 12, wherein the HBV neutralizing antibodies is administered to the patient by infusing one, two or three exogenous human HBV neutralizing antibodies at a dose of about 0.1 mg/kg body weight.

15. The method of claim 13, wherein infusion of exogenous human neutralizing antibodies comprising a single, multiple or repeated infusions in order to maintain a level of antibody at >1 mIU/ml in the patient's blood for three months or longer.

16. The method of claim 14, wherein HBV neutralizing antibodies is one, two or three endogenously expressed human neutralizing antibodies.

17. The method of claim 14, wherein endogenous human HBV neutralizing antibodies are expressed by viral or non-viral vectors that include nanoparticles like lipid nanoparticles (LNP) or GalNAc particles.

18. The methods of claim 16, wherein viral or non-viral vectors contains DNA or mRNA sequences which encode the HBV neutralizing antibodies or antibody fragments.

19. The method of claim 17, wherein viral vectors comprising an adeno-associated virus (AAV) vector.

20. The method of claim 18, wherein the AAV vectors comprise nucleic acid sequences encoding the HBV neutralizing antibodies or its antibody fragments and thus becomes AAV-anti-HBV vectors.

21. The method of claim 19, wherein the AAV-anti-HBV vectors are injected into muscle cells of HBV patients at a dose of about 1×10⁹ genome copies/kg; or higher.

22. The method of claim 20, wherein viral or non-viral vectors are injected to the patient at once or multiple times as needed.

23. The method of claim 21, wherein depleting rcDNA through a combination of HBV neutralizing antibody with intracellular HBV replication inhibitors lead to a significant, nearly complete, or complete cccDNA loss.

24. The method of claim 22, wherein the intracellular HBV inhibitors comprise RT inhibitors, capsid inhibitors, RNAi drugs, nucleic acid polymers (NAP), interferons, innate immunity agonists, and entry inhibitors.

25. The method of claim 23, wherein the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in chronic HBV infected people who are HBV treatment naïve.

26. The method of claim 24, wherein the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in chronic HBV infected people who are receiving HBV treatment but can't safely withdraw antiviral drugs.

27. The method of claim 24, wherein the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in chronic HBV infected people who wish to become HBV infection free regardless of their infection status/phase or their treatment status (naïve or treated).

28. The method of claim 24, wherein the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in HBV infected pregnant women or patients with liver transplant.

29. The method of claim 24, wherein the AAV anti-HBV vectors expressing HBV neutralizing antibodies is used for blocking cccDNA replenishment for reducing and eliminating cccDNA, for inducing HBsAg seroclearance, for realizing a complete anti-HBs seroconversion, and for a more effective HBV cure in people whose HBV infection has been clinically resolved but are at higher risk for HBV recurrence or reactivation.

30. An isolated binding molecule or antigen-binding fragment thereof which specifically binds to HBV virions and/or HBsAg subviral particles comprising an antibody VH, wherein the VH comprises the amino acid sequences of SEQ ID NO: 2 and an antibody VL, wherein the VL comprises the amino acid sequences of SEQ ID NO: 4, or a variant thereof with at least 95% sequence homology.

31. A nucleic acid molecule encoding the isolated binding molecule or antigen-binding fragment of claim 29.

32. A vector comprising the nucleic acid molecule of claim 30.

33. An isolated binding molecule or antigen-binding fragment thereof which specifically binds to HBV virions and/or HBsAg subviral particles comprising an antibody VH, wherein the VH comprises the amino acid sequences of SEQ ID NO: 6 and an antibody VL, wherein the VL comprises the amino acid sequences of SEQ ID NO: 8, or a variant thereof with at least 95% sequence homology.

34. A nucleic acid molecule encoding the isolated binding molecule or antigen-binding fragment of claim 32.

35. A vector comprising the nucleic acid molecule of claim 33.

36. An isolated binding molecule or antigen-binding fragment thereof which specifically binds to HBV virions and/or HBsAg subviral particles comprising an antibody VH, wherein the VH comprises the amino acid sequences of SEQ ID NO: 10 and an antibody VL, wherein the VL comprises the amino acid sequences of SEQ ID NO: 12, or a variant thereof with at least 95% sequence homology.

37. A nucleic acid molecule encoding the isolated binding molecule or antigen-binding fragment of claim 35.

38. A vector comprising the nucleic acid molecule of claim 36.

39. An isolated binding molecule or antigen-binding fragment thereof which specifically binds to HBV virions and/or HBsAg subviral particles comprising an antibody VH, wherein the VH comprises the amino acid sequences of SEQ ID NO: 14 and an antibody VL, wherein the VL comprises the amino acid sequences of SEQ ID NO: 16, or a variant thereof with at least 95% sequence homology.

40. A nucleic acid molecule encoding the isolated binding molecule or antigen-binding fragment of claim 38.

41. A vector comprising the nucleic acid molecule of claim 39.

42. An isolated binding molecule or antigen-binding fragment thereof which specifically binds to HBV virions and/or HBsAg subviral particles comprising an antibody VH, wherein the VH comprises the amino acid sequences of SEQ ID NO: 18 and an antibody VL, wherein the VL comprises the amino acid sequences of SEQ ID NO: 20, or a variant thereof with at least 95% sequence homology.

43. A nucleic acid molecule encoding the isolated binding molecule or antigen-binding fragment of claim 41.

44. A vector comprising the nucleic acid molecule of claim 42.

45. An isolated binding molecule or antigen-binding fragment thereof which specifically binds to HBV virions and/or HBsAg subviral particles comprising an antibody VH, wherein the VH comprises the amino acid sequences of SEQ ID NO: 22 and an antibody VL, wherein the VL comprises the amino acid sequences of SEQ ID NO: 24, or a variant thereof with at least 95% sequence homology.

46. A nucleic acid molecule encoding the isolated binding molecule or antigen-binding fragment of claim 44.

47. A vector comprising the nucleic acid molecule of claim 45.

48. An isolated binding molecule or antigen-binding fragment thereof which specifically binds to HBV virions and/or HBsAg subviral particles comprising an antibody VH, wherein the VH comprises the amino acid sequences of SEQ ID NO: 26 and an antibody VL, wherein the VL comprises the amino acid sequences of SEQ ID NO: 28, or a variant thereof with at least 95% sequence homology.

49. A nucleic acid molecule encoding the isolated binding molecule or antigen-binding fragment of claim 47.

50. A vector comprising the nucleic acid molecule of claim 48.

51. An isolated binding molecule or antigen-binding fragment thereof which specifically binds to HBV virions and/or HBsAg subviral particles comprising an antibody VH, wherein the VH comprises the amino acid sequences of SEQ ID NO: 30 and an antibody VL, wherein the VL comprises the amino acid sequences of SEQ ID NO: 32, or a variant thereof with at least 95% sequence homology.

52. A nucleic acid molecule encoding the isolated binding molecule or antigen-binding fragment of claim 50.

53. An isolated binding molecule or antigen-binding fragment thereof which specifically binds to HBV virions and/or HBsAg subviral particles comprising an antibody VH, wherein the VH comprises the amino acid sequences of SEQ ID NO: 34 and an antibody VL, wherein the VL comprises the amino acid sequences of SEQ ID NO: 36, or a variant thereof with at least 95% sequence homology.

54. A nucleic acid molecule encoding the isolated binding molecule or antigen-binding fragment of claim 52.

55. A vector comprising the nucleic acid molecule of claim 53.

56. A composition comprising the isolated binding molecule or antigen-binding fragment of claim 30.

57. A pharmaceutical composition comprising the antibody of claim 55 and a pharmaceutically acceptable carrier.

58. A method of treating chronic HBV infection and providing protection against new rounds of HBV infection in a human patient, comprising administering to the HBV infected human patient a sufficient amount of HBV neutralizing antibodies or antibody fragments, wherein said amount of HBV neutralizing antibodies is at a level which blocks replenishing both rcDNA and cccDNA, enabling reducing and depleting rcDNA to <1 copy/cell and facilitating reducing and eliminating cccDNA <1 copy/cell, inducing HBsAg seroclearance, realizing a complete anti-HBs seroconversion, and delivering a more effective HBV cure.

59. The method of claim 57, wherein the treating of chronic HBV infection comprises treating newborns/children who have been infected by HBV.

60. The method of claim 57, wherein the treating of chronic HBV infection comprises treating adults who have been infected by HBV.

61. The method of claim 57, wherein the HBV infected human patient is a chronic HBV infected individual who has been HBsAg positive for more than 6 months and have normal or elevated alanine aminotransferase (ALT) level.

62. The method of claim 57, wherein the HBV infected human patient is an HBV positive pregnant woman, or an organ transplant recipient who is HBsAg positive or HBsAg negative/anti hepatitis B core antibody (anti-HBc) positive and is prone to recurrent HBV infection after transplant.

63. The method of claim 57, wherein the HBV neutralizing antibodies or antibody fragments are produced by HBV therapeutic vectors.

64. The method of claim 62, wherein the HBV therapeutic vectors comprise a mixed population of vectors each of which encodes one specific anti-HBs antibody or antibody fragment binding to one or more epitopes of HBV envelope proteins, or a single vector which encodes one HBV neutralizing antibody or antibody fragment binding to one or more epitopes of HBV envelope proteins.

65. A composition comprising the isolated binding molecule or antigen-binding fragment of claim 33.

66. A composition comprising the isolated binding molecule or antigen-binding fragment of claim 36.

67. A composition comprising the isolated binding molecule or antigen-binding fragment of claim 39.

68. A composition comprising the isolated binding molecule or antigen-binding fragment of claim 42.

69. A composition comprising the isolated binding molecule or antigen-binding fragment of claim 45.

70. A composition comprising the isolated binding molecule or antigen-binding fragment of claim 48.

71. A composition comprising the isolated binding molecule or antigen-binding fragment of claim 51.

72. A composition comprising the isolated binding molecule or antigen-binding fragment of claim 53.
